SEQUENCE LISTING (32 sequences)

SEQ ID NO: 1 - protein, 111 aa, Artificial Sequence
  clone 33_VH1
EVQLVESGGGLVQPGGSLRLSCAASGFTFSSSAMTWVRQAPGKGLEWVASISGGGTTYYADSVKGRFTISRDNSKNTLYLQMNSLRAEDTAVYYCARSHYWGQGTLVTVSS

SEQ ID NO: 2 - protein, 108 aa, Artificial Sequence
  clone 33_VL4
DIQMTQSPSSLSASVGDRVTITCRASQNVVTNVAWYQQKPGKAPKALIYSASYLYSGVPSRFSGSGSGTDFTLTISSLQPEDFATYYCQQYSSYPYTFGGGTKVEIKR

SEQ ID NO: 3 - protein, 108 aa, Artificial Sequence
  clone 33_VL5
DIQMTQSPSSLSASVGDRVTITCRASQGVVTSVAWYQQKPGKAPKALIYSASYLYSGVPSRFSGSGSGTDFTLTISSLQPEDFATYYCQQYDSYPYTFGGGTKVEIKR

SEQ ID NO: 4 - protein, 457 aa, Artificial Sequence
  33_VH
MELGLSWIFLLAILKGVQCEVQLVESGGGLVQPGGSLRLSCAASGFTFSNSAMTWVRQAPGKRLEWVASISGGGTTYYPDSVKGRFTISRDNSKNTLYLQMNSLRAEDTAVYYCARSHYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 457 aa, Artificial Sequence
  33_VH1
MELGLSWIFLLAILKGVQCEVQLVESGGGLVQPGGSLRLSCAASGFTFSSSAMTWVRQAPGKGLEWVASISGGGTTYYADSVKGRFTISRDNSKNTLYLQMNSLRAEDTAVYYCARSHYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 457 aa, Artificial Sequence
  33_VH2
MELGLSWIFLLAILKGVQCEVQLVESGGGLVQPGGSLRLSCAASGFTFSNSAMNWVRQAPGKGLEWVASISGGGTTYYPDSVKGRFTISRDNSKNTLYLQMNSLRAEDTAVYYCARSHYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 457 aa, Artificial Sequence
  33_VH3
MELGLSWIFLLAILKGVQCEVQLVESGGGLVQPGGSLRLSCAASGFTFSSSAMSWVRQAPGKGLEWVATISGGGTTYYADSVKGRFTISRDNSKNTLYLQMNSLRAEDTAVYYCARSHYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 236 aa, Artificial Sequence
  33_VL
MDMRVPAQLLGLLLLWFPGARCDIQMTQSPSSVSASVGDRVTITCKASQNVVTNVAWYQQKPGKAPKALIYSASYRYSGVPSRFSGSGSGTDFTLTISSLQPEDFATYYCQQYNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 236 aa, Artificial Sequence
  33_VL1
MDMRVPAQLLGLLLLWFPGARCDIQMTQSPSSLSASVGDRVTITCRASQGVVTNVAWYQQKPGKAPKLLIYSASYRYSGVPSRFSGSGSGTDFTLTISSLQPEDFATYFCQQYNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 236 aa, Artificial Sequence
  33_VL2
MDMRVPAQLLGLLLLWFPGARCDIQMTQSPSSLSASVGDRVTITCRASQNVVTNVAWYQQKPGKAPKALIYSASYRQSGVPSRFSGSGSGTDFTLTISSLQPEDFATYFCQQYNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 236 aa, Artificial Sequence
  33_VL3
MDMRVPAQLLGLLLLWFPGARCDIQMTQSPSSLSASVGDRVTITCRASQGVVTNVAWYQQKPGKAPKLLIYSASYRQSGVPSRFSGSGSGTDFTLTISSLQPEDFATYFCQQYSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 236 aa, Artificial Sequence
  33_VL4
MDMRVPAQLLGLLLLWFPGARCDIQMTQSPSSLSASVGDRVTITCRASQNVVTNVAWYQQKPGKAPKALIYSASYLYSGVPSRFSGSGSGTDFTLTISSLQPEDFATYYCQQYSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 236 aa, Artificial Sequence
  33_VL5
MDMRVPAQLLGLLLLWFPGARCDIQMTQSPSSLSASVGDRVTITCRASQGVVTSVAWYQQKPGKAPKALIYSASYLYSGVPSRFSGSGSGTDFTLTISSLQPEDFATYYCQQYDSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 7 aa, Artificial Sequence
  CDR1
GFTFSSS

SEQ ID NO: 15 - protein, 7 aa, Artificial Sequence
  CDR2
GFTFSNS

SEQ ID NO: 16 - protein, 7 aa, Artificial Sequence
  CDR2
SGGGTTY

SEQ ID NO: 17 - protein, 11 aa, Artificial Sequence
  light chain CDR1
  MISC_FEATURE (5)..(5): is N or G
  MISC_FEATURE (9)..(9): is N or S
RASQXVVTXVA

SEQ ID NO: 18 - protein, 11 aa, Artificial Sequence
  Light chain CDR1
RASQNVVTNVA

SEQ ID NO: 19 - protein, 11 aa, Artificial Sequence
  Light chain CDR1
RASQGVVTSVA

SEQ ID NO: 20 - protein, 7 aa, Artificial Sequence
  Light chain CDR2
SASYLYS

SEQ ID NO: 21 - protein, 9 aa, Artificial Sequence
  Light chain CDR3
  MISC_FEATURE (4)..(4): is S or D
QQYXSYPYT

SEQ ID NO: 22 - protein, 5 aa, Artificial Sequence
  amino acid sequence
LFPAP

SEQ ID NO: 23 - protein, 108 aa, Artificial Sequence
  33_VL
DIQMTQSPSSLSASVGDRVTITCKASQNVVTNVAWYQQKPGKAPKALIYSASYRYSGVPSRFSGSGSGTDFTLTISSLQPEDFATYYCQQYNSYPYTFGGGTKVEIKR

SEQ ID NO: 24 - protein, 108 aa, Artificial Sequence
  33_VL_1
DIQMTQSPSSLSASVGDRVTITCRASQGVVTNVAWYQQKPGKAPKLLIYSASYRYSGVPSRFSGSGSGTDFTLTISSLQPEDFATYFCQQYNSYPYTFGGGTKVEIKR

SEQ ID NO: 25 - protein, 108 aa, Artificial Sequence
  33_VL_2
DIQMTQSPSSLSASVGDRVTITCRASQNVVTNVAWYQQKPGKAPKALIYSASYRQSGVPSRFSGSGSGTDFTLTISSLQPEDFATYFCQQYNSYPYTFGGGTKVEIKR

SEQ ID NO: 26 - protein, 108 aa, Artificial Sequence
  33_VL_3
DIQMTQSPSSLSASVGDRVTITCRASQGVVTNVAWYQQKPGKAPKLLIYSASYRQSGVPSRFSGSGSGTDFTLTISSLQPEDFATYFCQQYSSYPYTFGGGTKVEIKR

SEQ ID NO: 27 - protein, 108 aa, Artificial Sequence; identical to SEQ ID NO: 2
  33_VL_4

SEQ ID NO: 28 - protein, 108 aa, Artificial Sequence; identical to SEQ ID NO: 3
  33_VL_6

SEQ ID NO: 29 - protein, 111 aa, Artificial Sequence
  33_VH
EVQLVESGGGLVQPGGSLRLSCAASGFTFSNSAMTWVRQAPGKRLEWVASISGGGTTYYPDSVKGRFTISRDNSKNTLYLQMNSLRAEDTAVYYCARSHYWGQGTLVTVSS

SEQ ID NO: 30 - protein, 111 aa, Artificial Sequence; identical to SEQ ID NO: 1
  33_VH_1

SEQ ID NO: 31 - protein, 111 aa, Artificial Sequence
  33_VH_2
EVQLVESGGGLVQPGGSLRLSCAASGFTFSNSAMNWVRQAPGKGLEWVASISGGGTTYYPDSVKGRFTISRDNSKNTLYLQMNSLRAEDTAVYYCARSHYWGQGTLVTVSS

SEQ ID NO: 32 - protein, 111 aa, Artificial Sequence
  33_VH_3
EVQLVESGGGLVQPGGSLRLSCAASGFTFSSSAMSWVRQAPGKGLEWVASISGGGTTYYADSVKGRFTISRDNSKNTLYLQMNSLRAEDTAVYYCARSHYWGQGTLVTVSS

(19) United States
(12) Patent Application Publication
(10) Pub. No.: US 2021/0095011 A1
(43) Pub. Date: Apr. 1, 2021

(54) IMPROVED ANTI-FIBRONECTIN EDA ANTIBODIES

(71) Applicant: EnCare Biotech B.V., Utrecht (NL)

(72) Inventors: Anton Egbert Peter ADANG, Eindhoven (NL); Fatih ARSLAN, Utrecht (NL)

(21) Appl. No.: 17/051,804

(22) PCT Filed: May 1, 2019
(86) PCT No.: PCT/NL2019/050253

§ 371 Date: Oct. 30, 2020

(30) Foreign Application Priority Data
May 3, 2018 (EP) 18170693.8

(51) Int. Cl. C07K 16/18

(52) U.S. Cl. CPC C07K 16/18; A61K 2039/505; C07K 2317/21; C07K 2317/565

(57) ABSTRACT

The disclosure concerns antibodies that bind fibronectin-EDA. These antibodies are particularly useful for use in treatment, prevention, or prevention of progression of fibrosis, adverse cardiac remodeling and conditions resulting from or relating to myocardial infarction and pressure-overload, such as heart failure, aneurysm formation and remote myocardial fibrosis and for use in improving angiogenesis, preferably after ischemic injury.

### FIELD OF THE INVENTION

The disclosure concerns antibodies that bind fibronectin-EDA. These antibodies are particularly useful for the treatment, prevention, or prevention of progression of fibrosis, adverse cardiac remodeling and conditions resulting from or relating to myocardial infarction and pressure-overload, such as heart failure, aneurysm formation and remote myocardial fibrosis and for use in improving angiogenesis, preferably after ischemic injury.

### BACKGROUND OF THE INVENTION

Ischemic heart disease is the largest socio-economic burden to Western societies. The most severe and acute complication of ischemic heart disease is a heart attack, also known as myocardial infarction. In the USA, EU and Japan, 2.4 million patients suffer from a myocardial infarction each year. Complications after myocardial infarction such as heart failure, fibrosis and arrhythmia result in high mortality rates and morbidity. The most important determinant of these complications is an improper cardiac repair response, referred to as adverse (cardiac) remodeling or adverse ventricular remodeling.

Heart failure (HF) has gained much attention, as it is the most severe and most frequent consequence of adverse remodeling after myocardial infarction. In the USA, EU and Japan alone, at least 1.8 million patients are hospitalized with newly diagnosed infarction-related HF each year. The mortality rate is 20% within a year from diagnosis, while 50% of patients die within 5 years. Quality of life of those that survive is severely affected as they suffer from progressively decreasing exercise tolerance and reduced capacity to conduct normal daily activities.

Current therapy for myocardial infarction aims at restoring blood flow through the occluded coronary artery. Anti-thrombotics (i.e., agents preventing blood clot formation) together with stents are the most important drug and device classes to optimize blood flow restoration after myocardial infarction. Despite these advances in blood flow optimization, infarction-related complications still occur and are increasing. The main reason is the fact that adverse remodeling is a completely different pathophysiological process than blood flow restoration.

The healing of the infarcted heart is a complex process involving many types of cells. Myocardial infarction is an acute event in which part of the heart muscle dies resulting in loss of pump function. Immediately after this acute event, repair processes are induced in the blood and the heart muscle, characterized by enhanced inflammation. However, the type of inflammation determines whether the infarcted heart is repaired and remodeled properly. The key factor that drives improper healing and deleterious inflammation is the activation of innate immunity by molecules related to cardiac death and matrix degradation. As a result, the heart will enter a process called adverse remodeling. Adverse remodeling has several deleterious consequences: heart failure, dilatation and fibrosis of the heart, disturbed contractility and relaxation, and disturbed electrical activation are known complications. The increasing incidence of infarction-related morbidity, like heart failure, emphasizes the need for novel therapeutics to enhance cardiac repair after infarction. Another factor contributing to healing of the infarcted heart, in particular in the early stage following myocardial infarction, is angiogenesis. De novo formation of microvessels has the potential to recover ischemic myocardium at early stages after myocardial information, contributes to prevent the transition to heart failure.

The main determinant for leukocytes to cause a deleterious inflammatory reaction is the deposition of fibronectin-EDA. After myocardial infarction, fibronectin-EDA is newly synthesized and transiently upregulated in the infarcted myocardium. Fibronectin-EDA can activate the immune system and other cells involved in matrix turnover, thereby inducing the migration and differentiation of cells involved in cardiac repair (e.g. leukocytes, lymphocytes and fibroblasts). Subsequently, cells activated by fibronectin-EDA induce detrimental inflammatory reactions in the healing heart.

Cellular fibronectin is a multifunctional adhesive glycoprotein present in the ECM and is produced by cells in response to tissue injury as occurs with MI. It contains an alternatively spliced exon encoding type III repeat extra domain A (EIIIA; EDA), that acts as an endogenous ligand for both TLR2 and TLR4 and integrin α4β1, α4β7 and α9β1. Fibronectin-EDA is not normally expressed in healthy human tissue, but is highly upregulated in newly developing vasculature during embryogenesis and in several (pathological) conditions such as (cardiac) ischemic tissue, atherosclerotic lesions, fibrotic tissue, tumors, transplant rejection and wounds. Overexpression of EDA results in enhanced inflammation and injury after brain ischemia. Thus, fibronectin-EDA is capable of activating leukocytes and cause an upregulation of cytokines and chemokines. It was recently shown that fibronectin-EDA knockout mice exhibited reduced fibrosis, preserved cardiac function and reduced ventricular dilatation compared to wild-type mice after myocardial infarction (Arslan F. et al. Circ. Res., March 2011: 108: 582-592). The splice variant fibronectin-EDA is also found to be present in the deposited tissue and is a potential stimulating factor of collagen production (Bhattacharyya et al 2014). As used herein, fibrosis refers to the excess deposition of fibrous tissue or the process of connective tissue deposition in healing. WO2012/057613 describes that treatment of mice with antibodies directed to the EDA domain of fibronectin-EDA. The document describes that the antibodies prevent left ventricular dilatation in said mice and improve survival after myocardial infarction. WO2015/088348 describes that antibodies directed to the EDA domain of fibronectin-EDA can be used for treatment, prevention or prevention of progression of adverse cardiac remodeling and conditions resulting from or relating to myocardial infarction, and improves angiogenesis after tissue injury.

There remains a need in the art for antibodies that are capable of treating, preventing, or preventing the progression of myocardial infarction-related conditions in a subject and that increase the chance of survival of a subject after myocardial infarction. There also remains a need in the art for antibodies that are capable of treating, preventing, or preventing the progression of fibrosis in a subject. It is an object of the present invention to provide for such antibodies.

### SUMMARY OF THE INVENTION

In one aspect, the disclosure provides an anti-fibronectin-EDA antibody or antigen binding fragment thereof comprising a heavy chain variable region and a light chain variable region, wherein the heavy chain variable region comprises, a CDR1 having the sequence GFTFSSS or GFTFSNS; a CDR2 having the sequence SGGGTTY; and a CDR3 having the sequence SHY and wherein the light chain variable region comprises, a CDR1 having the sequence RASQZ₁VVTZ₂VA, wherein Z₁ is N or G, and Z₂ is N or S, preferably wherein Z₁ is N and Z₂ is N or Z₁ is G and Z₂ is S; a CDR2 having the sequence SASYLYS; and a CDR3 having the sequence QQYZ₃SYPYT, wherein Z₃ is S or D.

In some embodiments, the anti-fibronectin-EDA antibody or antigen binding fragment thereof has a heavy chain variable region comprising the sequence of SEQ ID 1. In some embodiments, the anti-fibronectin EDA antibody or antigen binding fragment thereof has a light chain variable region comprising the sequence of SEQ ID 2 or SEQ ID 3, more preferably SEQ ID 2. In some embodiments, the anti-fibronectin-EDA antibody or antigen binding fragment thereof specifically binds to an amino acid sequence LFPAP.

In some embodiments, the antibody comprises a constant region of a human antibody, preferably an IgG constant region, preferably wherein said constant region is a region that is deficient in complement activation, more preferably human IgG₄ constant region or a mutated human IgG₁ constant region.

The disclosure further provides one or more nucleic acid molecules encoding the antibody or antigen binding fragment thereof as disclosed herein. Also provided is a nucleic acid encoding a variable region as disclosed herein. In one aspect the disclosure provides a vector comprising a nucleic acid molecule as described herein.

The disclosure further provides a cell comprising and/or producing an antibody or antigen binding fragment thereof as disclosed herein, and/or comprising a nucleic acid molecule as disclosed herein and/or comprising a vector as disclosed herein, preferably wherein the cell is a hybridoma cell, a Chinese hamster ovary cell, an NS0 cell or a PER-C6™ cell. Preferably, the host cell is a mammalian, insect, plant, bacterial or yeast cell. Most preferably, the cell is a human cell. The disclosure further provides a cell culture comprising a cell as disclosed herein.

One aspect of the disclosure concerns a method for producing and/or purifying any of the said antibodies according or antigen binding fragments, preferably wherein the antibody or antigen binding fragment thereof is produced comprising culturing a cell culture as describes before and harvesting said antibody or antigen binding fragment thereof from said culture.

One aspect of the disclosure provides a pharmaceutical composition comprising an antibody or antigen binding fragment thereof, one or more nucleic acid molecules, or a vector, and/or a cell or cell culture as disclosed. In some embodiments, the pharmaceutical composition is for use in therapy. In some embodiments, said therapy is for the treatment, prevention, or prevention of the progression of fibrosis. In some embodiments, said therapy is for the treatment, prevention, or prevention of the progression of adverse cardiac remodeling, conditions resulting from or relating to myocardial infraction and/or pressure overload. Preferably, wherein said therapy is for improving angiogenesis.

In some embodiments, the composition or antibody or antigen binding fragment thereof as disclosed herein are for use in the manufacture of a medicament. Preferably, the medicament is for the treatment, prevention of prevention of the progression of adverse cardiac remodeling, conditions resulting from or relating to myocardial infarction and/or pressure overload. Preferably, the medicament is for the treatment, prevention, or prevention of the progression of fibrosis.

In some embodiments, methods are provided for treating an individual comprising administering to an individual in need thereof a therapeutically effective amount of a pharmaceutical composition or an antibody or antigen binding fragment thereof, as disclosed herein.

### BRIEF DESCRIPTION OF THE DRAWINGS

FIG. 1. Amino acid sequence alignment of variable domains

Amino acid sequence alignment of variable regions of both the light and the heavy chain compared to the variable regions of the Clone33 antibody. Differences in amino acid sequence are highlighted in gray or white, depending on the extent of the alteration at the amino acid level. Identical amino acids in the sequence are highlighted in black. CDRs are indicated in the figure according to the Chothia definition.

FIG. 2. ELISA to compare different antibody variants for Fibronectin-EDA binding.

Binding properties of variants 1-15 compared to clone 33 antibody. A) Shows the binding properties of the antibody variants to the EDA-fragment. B) shows the binding properties of the antibody variants to the EDA-peptide.

FIG. 3. ELISA variant 10 binding to EDA peptide or fragment.

Adhesion properties of variant 10 to fibronectin-EDA are measured in an ELISA assay. Variant 10 has similar binding properties for both the EDA fragment and EDA peptide in an ELISA assay as compared to the clone 33 antibody.

FIG. 4. Adhesion assay of variant 10 to EDA and control peptides.

Adhesion assay showing the specificity of binding of clone 33 and variant 10 to the EDA peptide (parental) compared to control peptides.

FIG. 5. ELISA variant 10 antibodies produced by stable cell lines.

Binding properties of antibodies produced by a subset of clonal cell lines producing variant 10 antibody.

FIG. 6 A-C. Adhesion properties of variant 10 antibodies produced by stable cell lines.

Adhesion assay showing the adhesion properties of variant 10 antibodies produced by a subset of clonal cell lines. Antibodies bind specific to EDA parental peptide over control peptides.

### DETAILED DESCRIPTION OF THE DISCLOSED EMBODIMENTS

The disclosure concerns antibodies that bind fibronectin EDA. Such antibodies bind the EDA domain of fibronectin-EDA. These antibodies are particularly useful in the treatment, prevention, or prevention of the progression of adverse cardiac remodeling, conditions resulting from or relating to myocardial infraction and/or pressure overload as well as for the treatment or prevention of fibrosis.

WO2015/088348 describes the anti-fibronectin EDA antibody 33E3.10 (also referred to herein as antibody 33). The present disclosure provides antibodies and antigen fragments thereof with improved characteristics for the expression and manufacture of anti-fibronectin EDA antibodies. Such characteristics may include for example, protein stability, yield, binding affinity, production cell viability, and reduced immunogenicity. Such characteristics are useful when manufacturing said antibodies or antigen binding fragments thereof at a large scale. Preferably, at least one of the characteristics is improved over the 33E3.10 antibody. In preferred embodiments, the antibodies and antigen fragments thereof provided herein exhibit reduced aggregation properties as compared to the 33E3.10 antibody, while maintaining good binding characteristics.

The term “fibronectin-EDA” as used herein refers to the extra domain A (EDA) of fibronectin. The entire fibronectin molecule is a glycoprotein present in the extra-cellular matrix. The EDA fragment arises from alternatively spliced transcripts and is produced during embryonic development. After the completion of development, fibronectin-EDA is produced as a result of tissue injury or other disease-related processes.

The term “antibody” as used herein refers to an immunoglobulin molecule that is typically composed of two identical pairs of polypeptide chains, each pair of chain consist of one “heavy” chain with one “light” chain. The human light chains are classified as kappa and lambda. The heavy chains comprise different classes namely: mu, delta, gamma, alpha or epsilon. These classes define the isotype of the antibody, such as IgM, IgD, IgG IgA and IgE, respectively. These classes are important for the function of the antibody and help to regulate the immune response. Both the heavy chain and the light chain consist of a variable and a constant region. The constant region of the heavy chain is clearly bigger than the constant region of the light chain, explaining the nomenclature of the heavy and light chain. Each heavy chain variable region (VH) and light chain variable region (VL) comprises complementary determining regions (CDR) interspersed by framework regions (FR). The variable region consists in total four FRs and three CDRs. These are arranged from the amino- to the carboxyl-terminus as follows: FR1, CDR1, FR2, CDR2, FR3, CDR3, FR4. The variable regions of the light and heavy chain together form the antibody binding site and defines the specificity for the epitope. The assignment of the amino acids to each region or domain of this disclosure is in accordance with the definitions of Chothia.

As used herein, antigen-binding fragments include Fab, F(ab′), F(ab′)₂, complementarity determining region (CDR) fragments, single-chain antibodies (scFv), bivalent single-chain antibodies, and other antigen recognizing immunoglobulin fragments. In some instances, the term “antibody” as used herein can be understood to also include an antigen binding fragment thereof.

The term “antibody” encompasses murine, humanized, deimmunized human and chimeric antibodies, and an antibody that is a multimeric form of antibodies, such as dimers, trimers, or higher-order multimers of monomeric antibodies. Antibody also encompasses monospecific, bispecific or multi-specific antibodies, and any other modified configuration of the immunoglobulin molecule that comprises an antigen recognition site of the required specificity. It also encompasses an antibody that is linked or attached to a non-antibody moiety. Further, the term “antibody” is not limited by any particular method of producing the antibody. For example, it includes monoclonal antibodies, recombinant antibodies and polyclonal antibodies.

The antibodies as disclosed herein may further comprise a moiety for increasing the in vivo half-life of the molecule, such as but not limited to polyethylene glycol (PEG), human serum albumin, glycosylation groups, fatty acids and dextran. Such further moieties may be conjugated or otherwise combined with the antibodies using methods well known in the art. In some embodiments, the antibodies as disclosed herein can be coupled to an active compound, for example a toxin. Furthermore, the antibodies or antigen binding fragments as disclosed may be coupled to a label, e.g. a fluorescent protein, chemical label, organic dye, colored particle or enzyme. The antibodies as disclosed herein can be coupled to a drug to form a antibody-drug conjugate (ADC).

Preferably, an antibody or antigen binding fragment thereof as disclosed herein is a humanized antibody or antigen binding fragment thereof. The term “humanized antibody” refers to an antibody that contains some or all of the CDRs from a non-human animal antibody while the framework and constant regions of the antibody contain amino acid residues derived from human antibody sequences. Humanized antibodies are typically produced by grafting CDRs from a mouse antibody into human framework sequences followed by back substitution of certain human framework residues for the corresponding mouse residues from the source antibody. The term “deimmunized antibody” also refers to an antibody of non-human origin in which, typically in one or more variable regions, one or more epitopes have been removed, that have a high propensity of constituting a human T-cell and/or B-cell epitope, for purposes of reducing immunogenicity. The amino acid sequence of the epitope can be removed in full or in part. However, typically the amino acid sequence is altered by substituting one or more of the amino acids constituting the epitope for one or more other amino acids, thereby changing the amino acid sequence into a sequence that does not constitute a human T-cell and/or B-cell epitope. The amino acids are substituted by amino acids that are present at the corresponding position(s) in a corresponding human variable heavy or variable light chain as the case may be. In some embodiments, an antibody or antigen binding fragment thereof as disclosed herein is a human antibody or antigen binding fragment thereof. The term “human antibody” refers to an antibody consisting of amino acid sequences of human immunoglobulin sequences only. Human antibodies may be prepared in a variety of ways known in the art.

In some embodiments, the antibody is a bispecific antibody. For such bispecific antibodies, one Fab fragment comprises the CDRs and/or variable regions described herein. The second Fab fragment may also recognize fibronectin EDA, or alternatively a second target.

In some embodiments, the antibody or antigen binding fragment thereof is an isolated antibody or antigen binding fragment thereof. The term “isolated” as used herein refer to material which is substantially or essentially free from components which normally accompany it in nature.

One aspect of the disclosure provides an anti-fibronectin-EDA antibody or antigen binding fragment thereof comprising a heavy chain variable region and a light chain variable region,

wherein the heavy chain variable region comprises
a CDR1 having the sequence GFTFSSS or GFTFSNS;
a CDR2 having the sequence SGGGTTY; and
a CDR3 having the sequence SHY
   and wherein the light chain variable region comprises
a CDR1 having the sequence RASQZ1VVTZ2VA, wherein Z is N or G, and Z2 is N or S, preferably wherein Z1 is N and Z2 is N or Z1 is G and Z2 is S;
a CDR2 having the sequence SASYLYS; and
a CDR3 having the sequence QQYZ3SYPYT, wherein Z3 is S or D.

Such antibodies differ from clone 33 in the at least the CDR3 region of the light chain variable region. Specifically, the asparagine is substituted with serine or aspartic acid.

Preferably, the CDR1 region of the heavy chain has the sequence GFTFSSS. Such antibodies differ further from antibody 33 as described in WO2015/088348 in the CDR1 region of the heavy chain variable region. Specifically, an asparagine is substituted with a serine residue.

The present disclosure provides a set of improved antibodies and antigen binding fragments thereof as compared to antibody 33. These antibodies are optimized to increase expression and decrease aggregation, while maintaining binding affinity.

The term “increased expression levels” as used herein refers to higher expression levels when the antibody or antigen binding fragment thereof is produced in a cell as compared to a similar cell producing the antibody 33 described in WO2015/088348. The cell may be a mammalian, insect, plant, bacterial or yeast cell. Examples of mammalian cell lines suitable include a hybridoma cell, a Chinese hamster ovary cell, an NSO cell, or a PER-C6™ cell. The cell may express the antibody or antigen binding fragment thereof as a result of the presence of vector encoding said antibody or antigen binding fragment thereof. Accordingly, the disclosure provides a vector comprising the nucleic acid molecule(s) as disclosed herein, which encodes the antibodies and antigen-binding fragments thereof as disclosed herein. For example, transfection of an expression vector results in the expression of the antibody or antigen binding fragment thereof in a cell. The yield and the titer of the antibody can be used to measure the levels of antibody expression.

The term “decrease aggregation” as used herein refers to higher levels of soluble antibody or antigen binding fragment thereof as compared to antibody 33 described in WO2015/088348. Especially, the amount of soluble antibody or antigen binding fragment thereof after protein purification is increased. Protein aggregation can be measured by methods known to the person skilled in the art. For example, using SE-HPLC or SDS page analysis can be used to analyze the aggregation profile.

An antibody or antigen binding fragment thereof according to the disclosure is preferably an antibody that is well tolerated in an animal and/or human. In particular, the antibodies have the same or preferably reduced immunogenicity in humans as compared to clone 33 described in WO2015/088348.

The term “immunogenicity” as used herein refers to the ability of a particular substance, such as an antigen, epitope or antibody, to provoke an immune response in the body of a human and other animal. Reduced immunogenicity refers to a reduced immune response in the body to the antibody or antigen binding fragment thereof as disclosed herein compared to antibody 33 described in WO2015/088348.

Preferably, the anti-fibronectin-EDA antibody or antigen binding fragment thereof specifically binds to an amino acid sequence LFPAP. The skilled person is aware of the meaning of the term ‘specifically binding’. The term “specifically binding” as used herein means that an Ig-like molecule or antibody or a fragment thereof as taught herein exhibits appreciable binding affinity for an antigen or a particular epitope and, preferably, does not exhibit significant cross-reactivity. An antibody that “does not exhibit significant cross-reactivity” is one that will not appreciably bind to an undesirable entity or tissue where fibronectin-EDA expression is absent. Specific binding can be determined according to any art-recognized means for determining such binding. For example, specific binding may be determined according to Scatchard analysis and/or competitive binding assays or other assays accepted in the field.

The disclosure further provides a heavy chain variable domain combined with a said light chain variable domain, as disclosed herein, in the form of a monoclonal antibody against fibronectin EDA. The antibody variable regions may be incorporated in a larger antibody molecule comprising, for example, a constant region of a human antibody. According to differences in their heavy chain constant domains, antibodies are grouped into five classes, or isotypes: IgG, IgA, IgM, IgD and IgE. These classes or isotypes comprise at least one of said heavy chains that is named with a corresponding Greek letter. In a preferred embodiment the disclosure provides an antibody according to the disclosure wherein said constant region is selected form the group of IgG, IgA, IgM, IgD and IgE constant regions, more preferably said constant region comprises an IgG constant region, more preferably an IgG constant region, preferably a mutated IgG₁ constant region, most preferably said constant region is an IgG₄ constant region. Furthermore, said IgG₄ constant region is preferably a human IgG₄ constant region. Preferably, the IgG₄ constant region of the disclosure comprises the constant regions of the heavy and light chain amino acid sequence. Some variations in the constant region of IgG₄ occurs in nature and/or is allowed without changing the immunological properties of the resulting antibody. Typically, between about 1-5 amino acid substitutions are allowed in the constant region. Such variants are also included in the scope of the disclosure. An antibody with an IgG₄ constant region or a mutated IgG₁ constant region has at least most of the pharmacological properties of an antibody but does not bind complement and will thus not induce depletion of the cells its binds to in vivo. Preferably said constant region is a constant region of a human antibody.

Preferably, said constant region is a region that is deficient in complement activation, preferably a human IgG₄ constant region or a mutated human IgG₁ constant region. The complement system is a part of the immune system that promotes inflammation, attacks the pathogens cell membrane and enhances the ability of antibodies and phagocytic cells to clear microbes and damaged cells.

In exemplary embodiments, the antibodies disclosed herein have a heavy chain variable region comprising the sequence of SEQ ID 1 and a light chain variable region comprising the sequence of SEQ ID 2 or SEQ ID 3. The disclosure further encompasses variants of said antibodies, in particular antibodies that differ at one or more positions of the framework regions. In some embodiments, it is possible to generate variants of an antibody or antigen binding fragment thereof as disclose herein by modifying one or more amino acids therein. Many of such variants will behave more or less similar when compared to said original. Such variants are also included in the scope of the disclosure. Preferably these variants have amino acid substitutions, insertions, deletions, or additions. Amino acid substitutions is the replacement of an amino acid with another amino acid. Preferably, the amino acid is preplaced by an amino acid having similar chemical properties, which is often called conservative substitution. Amino acid deletions result in the deletion of one or multiple amino acids form the sequence. Amino acid insertions result in one or more additional amino acids in the sequence. Amino acid additions result in one or more amino acids at the start or end of the amino acid sequence.

Fibronectin EDA binding by the antibodies and antigen binding fragments disclosed herein can be confirmed in a number of suitable assays known to the skilled person. Such assays include, e.g., affinity assays, e.g., western blots, radio-immunoassay, and ELISA (enzyme-linked immunosorbant assay). The examples describe in detail one of the many assays which can be used to measure fibronectin EDA binding.

In a further aspect, the disclosure provides nucleic acid molecules encoding said antibodies and antigen binding fragments. In the present invention, the terms “nucleic acid molecule,” or “polynucleotide molecule” are understood to refer to polymers of nucleotides of any length and include DNA and RNA. The nucleotides can be deoxyribonucleotides, ribonucleotides, modified nucleotides or bases, and/or their analogues, or any substrate that can be incorporated into a polymer by DNA or RNA polymerase, or by a synthetic reaction. Based on the genetic code, a skilled person can determine the nucleic acid sequence which encode the antibody variants disclosed herein. Based on the degeneracy of the genetic code, sixty-four codons may be used to encode twenty amino acids and translational terminal signal. As is known to a skilled person, codon usage bias in different organisms can affect gene expression level. Various computational tools are available to the skilled person in order to optimize codon usage depending on which organisms the desired nucleic acid will be expressed.

In one embodiment a cell is provided comprising an antibody or antigen binding fragment thereof and/or a nucleic acid according to the disclosure. The host cells may be a mammalian, insect, plant, bacterial or yeast cell. Said cell is preferably a animal cell, preferably a mammalian cell, most preferably a human cell. Examples of mammalian cell lines suitable as host cells include a hybridoma cell, a Chinese hamster ovary cell, an NSO cell, or a PER-C6™ cell. For the purpose of the disclosure a suitable cell is any cell capable of comprising and preferably of producing said antibodies and/or said nucleic acids. The disclosure further encloses cell cultures that comprise said cells.

In a further aspect, the disclosure provides a vector comprising the nucleic acid molecule(s) as taught herein, which is capable of encoding the antibodies and antigen-binding fragments thereof as taught herein. The term “vector” is well-known in the art and is understood to refer to a nucleic acid molecule capable of artificially carrying or transporting foreign genetic material (i.e. nucleic acid molecule) to which it has been linked, into another cell, where it can be replicated and/or expressed.

In an embodiment, certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, vectors may comprise promoters that are capable of directing the expression of genes to which they are operatively linked. Vectors may be “expression vectors”. Other types of vectors include cosmids and artificial chromosomes. Methods and standard protocols for the preparation of suitable vectors comprising nucleic acid molecules which are capable of encoding the antibodies or antigen-binding fragments thereof as taught herein are also well known to the skilled person.

The antibodies or antigen binding fragments thereof disclosed herein can be produced by any method known to a skilled person. In a preferred embodiment, the antibodies or antigen binding fragments thereof are produced using a cell, preferably wherein the cell is a hybridoma cell, a Chinese hamster ovary cell, an NS0 cell or a PER-C6™ cell. In a particular preferred embodiment said cell is a Chinese hamster ovary cell, preferably said cell is cultured in serum free medium. This includes harvesting said antibody or antigen binding fragment thereof from said culture. The antibody is preferably purified form the medium, preferably said antibody is affinity purified. Alternatively, said antibodies or antigen binding fragments thereof can be generated synthetically.

Various institutions and companies have developed cell lines for the large scale production of antibodies, for instance for clinical use. These cells are also used for other purposes such as the production of proteins. Cell lines developed for industrial scale production of proteins and antibodies are herein further referred to as industrial cell lines. Thus a preferred embodiment of the disclosure provides the use of a cell line developed for the large scale production of said antibodies or antigen binding fragments thereof. The examples describe the production of such a cell line.

An antibody or antigen binding fragment thereof according to the invention exhibits a number of activities that can be advantageously used in therapeutic and non-therapeutic uses. In particular, antibodies or antigen binding fragments thereof as disclosed herein are useful for the treatment of an individual. Preferably, the antibodies or antigen binding fragments thereof as disclosed herein are useful for the treatment, prevention, or prevention of the progression of fibrosis. Preferably, the antibodies or antigen binding fragments thereof are useful for the treatment, prevention, or prevention of the progression of adverse cardiac remodeling, conditions resulting from or relating to myocardial infarction and/or pressure overload. In some embodiments, the antibodies or antigen binding fragments thereof are preferably used in therapy, preferably human therapy. In some embodiments, an antibody or antigen binding fragment thereof as disclosed herein may be used for research purposes. For example, in in vitro experiments, cell culture, organotypic culture and in vivo models.

Fibrosis refers to the formation of excess fibrous connective tissue in an organ of tissue. Fibrosis usually occurs in response to damage to a tissue or organ, and results in scarring and thickening of the affected tissue. Fibrosis is in essence an exaggerated wound healing response and can interfere with normal organ or tissue function. Reduced levels of fibrosis due to treatment with an antibody or antigen binding fragment thereof as disclosed herein can lead to restored or improved organ or tissue function.

Myocardial infarction occurs when the blood flow decreases or stops to a part of the heart. The interrupted blood flow often results in damages to the heart muscle. The antibodies or antigen-binding fragments as taught herein may be administered to an individual having one or more signs or symptoms of myocardial infarction and/or heart failure, such as chest pain, dyspnea, edema and cardiomegaly.

Adverse cardiac remodeling refers to changes in the size, shape, structure, and function of the heart. This can happen after injury of the heart muscle, for example after acute myocardial infarction. Cardiac remodeling may also result from increased pressure or volume in the heart, also called pressure overload or volume overload. Remodeling may result in reduced heart function and/or reduced contractile function of the heart muscle

Pressure overload refers to the pathological state of cardiac muscle in which it has to contract while experiencing an excessive afterload. Pressure overload may affect any of the four chambers of the heart, though the term is most commonly applied to one of the two ventricles. Pressure overload can be caused by an obstruction of the outflow of one of the chambers of the heart. Chronic pressure overload leads to initial concentric hypertrophy of the cardiac muscle and eventually dilatation due to adverse remodeling caused by prolonged pressure overload. In turn, this will lead to heart failure, myocardial ischaemia, and lethal arrhythmias.

Without being bound by theory, it is believed that fibronectin-EDA is deposited in the extra-cellular matrix of tissue damaged by, e.g. myocardial infarction, adverse cardiac remodeling and/or pressure overload.

The disclosure further comprises a pharmaceutical composition comprising an antibody or antigen binding fragment as disclosed herein, or a nucleic acid encoding same, or a cell comprising an antibody or antigen binding fragment as disclosed herein, or a nucleic acid encoding same. Such compositions are especially suited for use as a medicament. The compositions may be in any suitable forms, such as liquid, semi-solid and solid dosage forms.

Preferably, the pharmaceutical composition comprises a pharmaceutically acceptable diluent or carrier. As used herein, the term “pharmaceutically acceptable” refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio. Furthermore, the term “pharmaceutically acceptable diluent or carrier” refers to a pharmaceutically acceptable material, composition or vehicle, such as a liquid or solid filler, diluent, excipient, solvent or encapsulating material, involved in carrying or transporting the subject chemical from one organ, or portion of the body, to another organ, or portion of the body. Other examples of materials widely used in medicine are stents, included but not limited to polymer-based or absorbable (i.e. biodegradable) stents. In the art, these stents are called drug-eluting stents. In the present invention, the stents are covered with or include the pharmaceutical composition in order to have the pharmaceutical composition released to the site of interest (e.g. coronary arteries in case of myocardial infarction, carotid artery or its distal branches in case of ischemic brain injury/stroke).

In some embodiments, the pharmaceutical composition is for use in therapy, Preferably the therapy is for the treatment and prevention, or prevention of the progression of fibrosis. Preferably, the therapy is for the treatment, prevention, or prevention of the progression of adverse cardiac remodeling. Preferably the therapy is for the treatment and prevention, or prevention of the progression of conditions resulting from or relating to myocardial infarction and/or pressure overload. Preferably the therapy is for stimulating or improving angiogenesis.

Angiogenesis refers to a physiological process through which new blood vessels are formed refers. This includes the de novo formation of microvessels. Angiogenesis is involved in growth, development and wound healing. Angiogenesis is a factor that can contribute to the healing of the infarcted heart, in particular to the early stage following myocardial infarction. The term “improving angiogenesis” as used herein refers not only to stimulating angiogenesis but also to reducing or inhibiting the inhibition of angiogenesis.

In some embodiments, methods are provided for treating an individual comprising administering to an individual in need thereof a therapeutically effective amount of a pharmaceutical composition or an antibody or antigen binding fragment thereof, as disclosed herein. In some embodiments, methods are provided for improving angiogenesis in an individual in need thereof. The term “individual” as used herein refers to an animal and may be used in both human and veterinary treatments. In particular the animal is a vertebrate (e.g., a mammal or bird). Preferably, an individual is a mammal such as a primate, dog, mouse, or human. More preferably, the individual is a human. In some embodiments, the individual has suffered from or is at risk of suffering from a myocardiac infarction. For example, an individual may be treated prophylactically during or before cardiac or thoracic aortic surgery. In some embodiments, the individual has suffered from or is at risk of suffering from a fibrotic disorder. In some embodiments, the individual is suffering, has suffered from or is at risk of suffering from adverse cardiac remodeling. In some embodiments, the individual is suffering, has suffered or is at risk of suffering from pressure overload.

The pharmaceutical composition may be administered by any suitable routes and mode. As will be appreciated by the person skilled in the art, the route and/or mode of administration will vary depending upon the desired results. The pharmaceutical compositions may be formulated in accordance with routine procedures for administration by any routes, such as parenteral, topical, oral, sublingual, transdermal, or by inhalation or via drug-eluting stents. The compositions may be in the form of tablets, capsules, powders, drug-eluting stents, granules, lozenges, creams or liquid preparations, such as sterile parenteral solutions or suspensions or in the form of a spray, aerosol or other conventional method for inhalation. The pharmaceutical compositions of the present invention include those suitable for oral, nasal, topical (including buccal and sublingual), rectal, vaginal and/or parenteral administration.

In an embodiment, the pharmaceutical composition is administered parenterally. The phrases “parenteral administration” and “administered parenterally” as used herein mean modes of administration other than enteral and topical administration, usually by injection, and include, without limitation, intravenous, intramuscular, intraarterial, intracoronary, intrathecal, intracapsular, intraorbital, intracardiac, intradermal, intraperitoneal, transtracheal, subcutaneous, subuticular, intraarticular, subcapsular, subarachnoid, intraspinal, epidural and intrasternal injection and infusion.

Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, (b) the limitations inherent in the art of compounding such an active compound for the treatment of sensitivity in individuals, and (c) duration and level of expression of fibronectin-EDA in the related disease entity. For example, fibronectin-EDA expression reaches a peak at 2 to 3 weeks and reduces to baseline levels 5 to 6 weeks after acute myocardial infarction. Depending on the half-life of the anti-fibronectin-EDA antibodies or antigen-binding fragment thereof as taught herein, the compound will be administered once, twice, three times or more frequent if desired to cover the entire expression duration of fibronectin-EDA.

Actual dosage levels of the pharmaceutical compositions of the present invention may be varied so as to obtain an amount of antibodies or antigen-binding fragments thereof which is effective (“effective amount”) to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts. The dosage and scheduling for the formulation, which is selected can be determined by standard procedures, well known by a skilled person. Such procedures involve extrapolating and estimating dosing schedule form animal models, and then determining the optimal dosage in a human clinical dose ranging study. The dosage in pharmaceutical compositions will vary depending upon an number of factors, such as the desired release and pharmacodynamic characteristics.

In an embodiment, an effective amount of the antibody, or antigen-binding fragment thereof as taught herein, such as a monoclonal antibody, may be in the range of about 0.1 μg/kg to about 10 g/kg, such as about 1 μg/kg to about 1 g/kg, about 10 μg/kg to about 100 mg/kg, or about 0.1 mg/kg to about 50 mg/kg.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. As used herein, the terms “treating”, “preventing”, or “preventing progression of” are understood to not only encompasses preventing, e.g., the onset of adverse cardiac remodeling, but also encompasses the situation in which adverse cardiac remodeling has commenced but its progression is inhibited or reduced.

All patent and literature references cited in the present specification are hereby incorporated by reference in their entirety.

### EXAMPLES

### Example 1

In silico antibody engineering of Clone 33 variants

In silico protein engineering tools and know-how to reduce the aggregation propensity of the antibody ENC-001-1. The antibody is also known as “clone 33”, “33VH2VL2”, and “33”. Antibody “33” is used interchangeably with ENC-001-1 in figures and tables. Sequence analysis of ENC-001-1 was performed in conjunction with analysis of the mouse chimeric antibody and the CRO humanised variants. A structural homology model of the Fv-region was constructed using a molecular modelling platform. The variable domains were analysed for back-mutations or alternative substitutions that would restore the stability of the chimeric antibody. Lonza's Antibody Aggregation platform was utilised to screen ENC-001-1 and potential variants for substitutions which are predicted to reduce the aggregation score. All substitutions were evaluated in the homology model for their potential impact on binding affinity.

A total of 15 variants have been engineered.

### Methods

### Sequence Annotation

Positions and substitutions are specified according to their ordinal number in the sequence. The updated Chothia CDR definition (Al-Lazikani et al. 1997) will be used as reference.

### Sequence Alignments

Multiple alignments of the ENC-001-1 sequence to the mouse and human germline sequences were generated and entries in each alignment were ordered according to the sequence identity (SeqId) to the ENC-001-1 sequence. Reference sets were reduced to a unique set of sequences by clustering at 100% SeqId and excluding redundant entries.

### Antibody Aggregation

The antibody aggregation platform used in this study was developed using a machine learning algorithm based on sequence and structural features of antibodies (Obrezanova et al. 2015). The predictive aggregation model was trained and tested on a set of antibodies, designed to cover a wide chemical space and to contain low and high expressing as well as aggregating and non-aggregating antibodies. The characteristics of all antibodies in the set were experimentally determined in-house. The algorithm gives a categorical output of high or low risk of aggregation; antibodies in the higher category have an increased risk of aggregation above 5% after protein purification.

In addition to the high or low aggregation risk categorization the antibody aggregation platform generates a certainty score which can be used to compare the aggregation propensity of related antibodies.

### Identification of Residues at Critical Positions

Antibody Fv's have a number of critical positions that make up the VH/VL inter chain interface or are responsible for the discrete set of canonical structures that has been defined for 5 of the CDRs (Chothia and Lesk 1987, Al Lazikani et al. 1997); these positions should be considered in detail before substitutions are proposed for them. Table 1 and Table 2 below show the conserved positions within the VH/VL interface and the positions that determine the CDR canonical class (respectively), with numbering according to the Chothia definition.

TABLE 1
Conserved positions within the VH/VL interface

| Domain | Positions |
|---|---|
| VL | 34, 36, 38, 43, 44, 46, 87, 88, 89, 91, 96, 98 |
| VH | 35, 37, 39, 45, 47, 91, 93, 95 100-100K*, 101, 103 |

All positions are according to Chothia numbering
*The numbering of the position one N-terminal to position 101 differs by CDR H3 length

TABLE 2
Positions determining CDR canonical classes

| CDR | Key Residues |
|---|---|
| L1 | 2, 25,29, 30, 30D*, 33, 71 |
| L2 | 34 |
| L3 | 90, 94, 95, 97 |
| H1 | 24, 26, 29, 34, 94 |
| H2 | 54, 55, 71 |

All positions are according to Chothia numbering
*If CDR L1 is long enough to contain the position

### Construction of 3D Models

Structural models of the Fv-region for antibody ENC-001-1, and variants thereof, were generated using a modelling platform. Candidate structural template fragments for the framework (FR) and CDRs as well as the full Fv were scored, ranked and selected from an in-house antibody database based on their sequence identity to the target, as well as qualitative crystallographic measures of the template structure, such as the resolution (in Ångstöm (Å)).

### Post-Translational Modifications

Post-translational modifications (PTMs) can cause problems during the development of a therapeutic protein such as increased heterogeneity, reduced bioactivity, reduced stability, immunogenicity, fragmentation and aggregation. The potential impact of PTMs depends on their location and in some cases on solvent exposure. The sequences were analysed for the following potential PTMs: Asparagine deamidation, Aspartate isomerisation, C-terminal Lysine clipping, free Cysteine thiol groups, N- and O-glycosylation, N-terminal cyclisation, oxidation and pyroglutamate formation.

### Assessment of Potential Substitutions

All positions in the variable domains were assessed based on their potential impact on binding affinity and stability. Each position was classified as either: Neutral, Critical or Contributing.
Neutral—a substitution to another amino acid at this position should not affect binding affinity or stability.
Contributing—a substitution can be made but the position may be contributing to the binding affinity or stability. Retention of the Parental amino acid at this position should be considered.
Critical—the position must retain the Parental amino acid or risk a decreased binding affinity or reduced stability.

Critical positions are initially defined as those in the Chothia CDRs, determined to be at critical positions in the VH/VL interface (Table 1); at positions that help determine the CDR conformation (Table 2) or that are highly conserved in the reference alignment.

Neutral substitutions are generally solvent exposed positions in the framework and more than 5 Å from any side chain atoms of any CDR residues, residues within this region are classed as Contributing to the affinity. Contributing positions may be substituted, and in many cases this is done in order to efficiently engineer the antibody. The risk category of all positions is continually re-evaluated in the context of other substitutions.

Many positions are conserved and will only accept a small set, or only one, type of amino acid. Other positions are more variable and if they are found to be solvent exposed and remote to the CDRs then they can support almost any substitution.

### Results

ENC-001-1 is also known as “33” and is composed of the light chain 33VL2 and heavy chain 33VH2. ENC-001-1 is an IgG4/kappa antibody with stabilising H:S218P substitution in the IgG4 heavy chain and two additional Fc substitutions, H:F225A and H:L226A, to reduce antibody-dependent cellular cytotoxicity (ADCC).

### Sequence Analysis

Analysis of the domain content of ENC-001-1 showed it to be a full length IgG4/kappa antibody with stabilising H:S218P hinge substitution in the IgG4 heavy chain and two additional Fe substitutions, H:F225A and H:L226A, to reduce antibody-dependent cellular cytotoxicity (ADCC). The variable domains were isolated and annotated with Chothia CDR definitions and numbering

### Post-Translational Modifications

Post-translational modifications (PTMs) can cause problems during the development of a therapeutic protein such as increased heterogeneity and in some instances reduced bioactivity or reduced stability. PTMs located in the CDRs are of particular concern for antibodies as the modification can alter the bioactivity. Two potential PTMs in the CDRs were highlighted as Developability Risks before. The two deamidation sites as described in Table 3.

TABLE 3
Potential post-translational modifications of note

| | | Amino | |
|---|---|---|---|
| | | acid and | |
| Chain | Region | Position | Description |
| L | L3 | L:Asn92 | CDR L3 Asparagine with deamidation |
| | | | potential. PTM at this position has |
| | | | the potential to affect binding. |
| | | | Engineering the site to remove the |
| | | | PTM potential is recommended despite |
| | | | the fact that this position is |
| | | | frequently observed in contact with |
| | | | the antigen. An alternative mitigation |
| | | | strategy is monitoring for the presence |
| | | | of the PTM and process control. |
| H | H1 | H:Asn31 | CDR H1 Asparagine with deamidation |
| | | | potential. PTM at this position has |
| | | | the potential to affect binding. |
| | | | Engineering the site to remove the |
| | | | PTM potential is recommended despite |
| | | | the fact that this position is |
| | | | frequently observed in contact with |
| | | | the antigen. An alternative mitigation |
| | | | strategy is monitoring for the presence |
| | | | of the PTM and process control. |

### Asparagine Deamidation

The hydrolysis of the amide group on the side-chain of Asparagine, deamidation, is a non-enzymatic reaction that over time produces a heterogeneous mixture of Asparagine, isoAspartic acid and Aspartic acid at the effected position. In addition to causing charge heterogeneity, Asparagine deamidation can affect protein function if it occurs in a binding interface such as in antibody CDRs (Harris et al. 2001). The occurrence of deamidation is heavily influenced by pH and process conditions. Careful tuning of process parameters and formulation can usually be used to minimise the risk.

### Antibody Engineering

Two approaches were used in designing variants with a reduced aggregation propensity. The first focused on analysing positions with unusual residues and those that were retained from the chimeric mouse antibody. The second focused on changing the aggregation risk class from High to Low as predicted by Lonza's antibody aggregation platform. It was in general not possible to recommend potentially stabilising substitutions which were not at risk of affecting the binding affinity. The substitutions arising out of the two approaches are labelled “alternative residue” or “aggregation-focused”, respectively, in the tables describing the chains and variants below (Tables 4-7).

Each position was screened with all possible amino acid substitutions using Lonza's Antibody Aggregation platform and the results recorded. The assessment of each position was updated as work progressed to reflect a substitution's potential impact on aggregation and PTMs as well as sequence and structural analysis.

The final proposed substitutions and their effects are described in Table 4 and Table 5 for the light and heavy chains respectively.

TABLE 4
Light Chain Antibody Engineering Substitutions

| Region | Substitution | Description |
|---|---|---|
| FR1 | L:V11L | The humanising substitution from Methionine to Valine |
| | | is acceptable. A substitution to Leucine can be evaluated |
| | | for reduced aggregation propensity. Alternative residue |
| | | and aggregation focussed. |
| L1 | L:K24R | CDR L1 conservative substitution from Lysine to |
| | | Arginine reduces the aggregation propensity. Low risk of |
| | | affecting binding affinity. Included in both alternative |
| | | residue and aggregation-focussed sets. |
| L1 | L:N28G | Glycine is common at this position, Asparagine is not. |
| | | The position is rarely involved in antigen interactions, a |
| | | substitution should be possible. Evaluate a substitution |
| | | from Asparagine to Glycine in some engineered variants. |
| | | Alternative residue, included in one aggregation-focussed |
| | | sequence. |
| L1 | L:N32S | CDR L1 position predicted to reduce aggregation |
| | | propensity. Due to the position's potential involvement |
| | | both in antigen binding and VH/VL interactions the |
| | | substitution should be considered as a final option in the |
| | | list of substitutions to consider. Aggregation-focussed. |
| FR2 | L:A46L | Position part of the VH/VL interface. Analysis of the CRO |
| | | reports and humanised variants show that a substitution |
| | | from Alanine to Leucine reduces the binding affinity. |
| | | Leucine is a conserved residue at this position and may |
| | | be important for stability. Evaluate a substitution from |
| | | Alanine to Leucine in two engineered light chains, |
| | | accepting the risk of reduced binding affinity. Alternative |
| | | residue. |
| L2 | L:R54L | The CRO humanised variants retain the Parental |
| | | Arginine at this position. Leucine is more common |
| | | amongst the most similar human germlines. A |
| | | substitution to Leucine is predicted to reduce the |
| | | aggregation propensity. Evaluate a substitution from |
| | | Arginine to Leucine. Aggregation-focussed. |
| L2 | L:Y55Q | Position can be involved in antigen binding but is usually |
| | | not. An aggregation propensity reducing substitution |
| | | from Tyrosine to Glutamine should be evaluated. |
| | | Alternative residue and aggregation-focussed. |
| FR3 | L:Y87F | Conserved Tyrosine, part of the VH/VL interface. |
| | | Structural analysis of the homology model of the chimeric |
| | | Fv indicated that Phenylalanine may have a different |
| | | conformation compared to Tyrosine at this position. |
| | | Alternative residue. |
| L3 | L:N92S, | CDR L3 Asparagine with deamidation potential. A |
| | L:N92D | substitution from Asparagine to Serine is evaluated to |
| | | remove the deamidation site. A substitution to Aspartic |
| | | acid is predicted to reduce the aggregation propensity. |
| | | Substitutions in CDR L3 can potentially affect antigen |
| | | binding. L:N92S in both alternative residue and |
| | | aggregation-focussed set, L:N92D aggregation-focussed. |

TABLE 5
Heavy Chain Antibody Engineering Substitutions

| Region | Substitution | Description |
|---|---|---|
| H1 | H:N31S | CDR H1 Asparagine with deamidation potential. |
| | | Removal of the predicted PTM by substitution to Serine |
| | | is evaluated in two heavy chains. The position may be |
| | | involved in antigen binding. Alternative residue and |
| | | aggregation-focussed sets. |
| FR2 | H:T35S, | Buried position, influences CDR conformation. |
| | H:T35N | Substitutions from Threonine to Serine and Asparagine |
| | | are predicted to reduce aggregation propensity. |
| | | Asparagine is predicted to reduce aggregation propensity |
| | | more than Serine. However, Serine is more similar to |
| | | Threonine and less likely to affect binding affinity. |
| | | Substitutions at this position can affect binding affinity. |
| | | H:T35S alternative residue, H:T35N aggregation- |
| | | focussed. |
| FR2 | H:R44G | Arginine from the mouse chimeric sequence has been |
| | | retained. The CRO humanised heavy chains show no |
| | | clear preference for Arginine or Glycine at this position. |
| | | The second humanisation evaluation round only |
| | | evaluates Glycine in one variant and it has an |
| | | acceptable, but not the best, binding affinity. The position |
| | | is a conserved Glycine in human germlines. Evaluate |
| | | stabilising the antibody by an Arginine to Glycine |
| | | substitution. Adternative residue and aggregation- |
| | | focussed sets. |
| FR2 | H:S50T | Position is frequently found in antigen contact. A |
| | | substitution from Serine to Threonine can be performed |
| | | at this position in concert with the substitution H:T35S. |
| | | Alternative residue. |
| FR3 | H:P60A | Unusual Proline in framework. A substitution to Alanine |
| | | can be evaluated. Alternative residue. |

### Engineered Variants

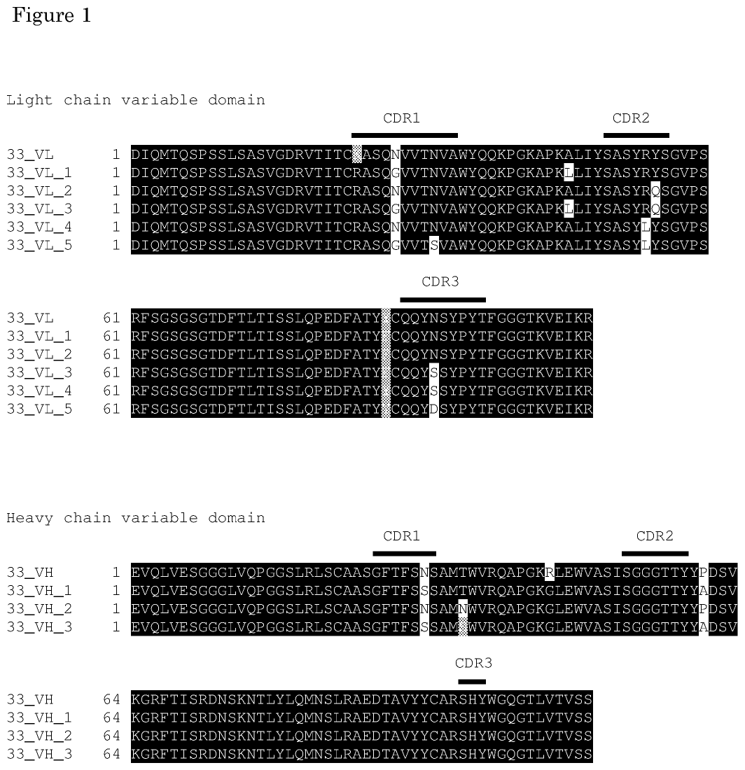

In order to attempt to evaluate the impact of all substitutions in an effective manner the substitutions have been grouped where it is appropriate to do so. A total of five engineered light chains and three engineered heavy chains have been proposed. Substitutions removing the predicted PTMs were incorporated into three light chains and two heavy chains. Table 6 lists the name of the engineered chains along with a description of the modifications. The amino acid sequences of the engineered chains are available in FIG. 1.

TABLE 6
Engineered chains

| Chain | Name | Description |
|---|---|---|
| L | 33_VL | ENC-001-1 Parental light chain |
| L | 33_VL_1 | Evaluating alternative residues L:V11L, L:K24R, |
| | | L:N28G, L:A46L, L:Y87F. L:Ala46 position is a |
| | | conserved Leucine which could be important for |
| | | stability. This substitution is known to affect binding |
| | | affinity. |
| L | 33_VL_2 | Evaluating alternative residues L:V11L, L:K24R, |
| | | L:Y55Q, L:Y87F. |
| L | 33_VL_3 | Evaluating alternative residues L:V11L, L:K24R, |
| | | L:N28G, L:A46L, L:Y55Q, L:Y87F and L:N92S. |
| | | L:N92S removes the deamidation site in CDR L3. |
| L | 33_VL_4 | Aggregation score focussed engineered sequence |
| | | with ubstitutions L:V11L, L:K24R, L:R54L and |
| | | L:N92S. L:N92S removes the deamidation site in |
| | | CDR L3. |
| L | 33_VL_5 | Aggregation score focussed engineered sequence |
| | | with substitutions L:V11L, L:K24R, L:N28G, |
| | | L:N32S, L:R54L and L:N92D. L:N92D removes the |
| | | deamidation site in CDR L3. |
| H | 33_VH | Parental heavy chain |
| H | 33_VH_1 | Evaluating alternative residues H:N31S, H:R44G, |
| | | H:P60A. H:N31S removes deamidation site in |
| | | CDR H1. |
| H | 33_VH_2 | Aggregation score focussed engineered sequence |
| | | with substitutions H:T35N and H:R44G. |
| H | 33_VH_3 | Combination of evaluating alternative residue, |
| | | removing deamidation site and aggregation score |
| | | focussed substitutions. |

In order to evaluate the impact of all substitutions in an effective manner an experimental design of variant combinations has been recommended in Table 7.

TABLE 7
Combinations of Heavy and Light chains used

| | | Light chains | | | | | |
|---|---|---|---|---|---|---|---|
| Combinations | | VL | VL1 | VL2 | VL3 | VL4 | VL5 |
| Heavy | VH | Clone 33 | | | | | |
| chains | VH1 | | 33_var1 | 33_var4 | 33_var7 | 33_var10 | 33_var13 |
| | VH2 | | 33_var2 | 33_var5 | 33_var8 | 33_var11 | 33_var14 |
| | VH3 | | 33_var3 | 33_var6 | 33_var9 | 33_var12 | 33_var15 |

### Antibody Aggregation Results

The Antibody Aggregation prediction results for Parental ENC-001-1 and the engineered variants are given in Table 8. The platform predicts whether the antibody is in a Low or High Aggregation Risk Class. The aggregation score is related to the class with positive scores indicating High Risk Class and negative scores Low Risk Class. The absolute value of the Aggregation Score indicates an increased certainty in the prediction. Hence, amore negative Aggregation Score compared to the Parental is sought in this project. The ΔScore indicates the change from the Parental antibody, with amore negative score being preferable.

TABLE 7
Antibody Aggregation Results

| Variant Name | Risk Class | Aggregation Score | ΔScore |
|---|---|---|---|
| 33 (ENC-001-1) | High | 2.6 | — |
| 33_var1 | High | 2.8 | 0.2 |
| 33_var2 | High | 1.2 | −1.3 |
| 33_var3 | High | 2.2 | −0.3 |
| 33_var4 | High | 3.1 | 0.6 |
| 33_var5 | High | 1.6 | −1.0 |
| 33_var6 | High | 2.6 | 0.0 |
| 33_var7 | High | 3.1 | 0.6 |
| 33_var8 | High | 1.6 | −1.0 |
| 33_var9 | High | 2.6 | 0.0 |
| 33_var10 | High | 2.1 | −0.5 |
| 33_var11 | High | 0.5 | −2.1 |
| 33_var12 | High | 1.5 | −1.0 |
| 33_var13 | Low | −0.1 | −2.7 |
| 33_var14 | Low | −1.7 | −4.3 |
| 33_var15 | Low | −0.7 | −3.2 |

ΔScore = Parental 33 Score − Variant Score

### Example 2. Transient Expression of Clone 33 Variants

Expression of 15 recombinant monoclonal antibody variants of ENC-001-1, alongside the parental monoclonal antibody ENC-001/1_WT, in Chinese Hamster Ovary cells (CHOK1SV GS-KO) using small scale transient expression, followed by Protein A part-purification and product quality analysis.

Single gene vectors were established for each heavy chain and light chain. The products were progressed to transient transfections in CHOK1SV GS-KO cells using the established single gene vectors (SGVs) to express the products for the assessment of the purification strategy (Protein A) and product quality by SDS-PAGE, SE-HPLC and endotoxin testing.

Each variant was transfected into CHOK1SV GS-KO cells and cultured for a set period. Cultures were harvested on day 6 and the supernatant was clarified by centrifugation followed by filter sterilisation using a 0.22 gm filter cartridge. Protein A purification was performed using clarified supernatant. Product quality analysis in the form of SE-HPLC, SDS-PAGE and endotoxin detection was carried out using purified material at 1 mg/ml.

A summary of the yields and product quality analysis are shown in Table 9.

TABLE 9
Yields and titres of small scale expression cultures

| | | Concentration | Volume | Yield | Titre | Monomer | Endotoxin |
|---|---|---|---|---|---|---|---|
| Product | Lot # | (mg/ml) | (ml) | (mg) | (mg/L) | (%) | (EU/mg) |
| ENC- | 360- | 0.053 | 20 | 1.06 | 2.65 | 68.92 | 4.12 |
| 001/1_WT | 270416- | | | | | | |
| ENC-001-1 | 360- | 0.09 | 40 | 3.6 | 9 | 96.38 | 7.25 |
| varl | 260416-1 | | | | | | |
| ENC-001-1 | 360- | 0.082 | 25 | 2.05 | 5.1 | 97.08 | 0.430 |
| var2 | 260416-2 | | | | | | |
| ENC-001-1 | 360- | 0.098 | 25 | 2.45 | 6.1 | 97.8 | 0.378 |
| var3 | 260416-3 | | | | | | |
| ENC-001-1 | 360- | 0.178 | 30 | 5.34 | 13.3 | 96.7 | 0.477 |
| var4 | 260416-4 | | | | | | |
| ENC-001-1 | 360- | 0.081 | 20 | 1.62 | 4 | 98.5 | 0.489 |
| var5 | 260416-5 | | | | | | |
| ENC-001-1 | 360- | 0.096 | 20 | 1.92 | 4.8 | 98.08 | 0.430 |
| var6 | 260416-6 | | | | | | |
| ENC-001-1 | 360- | 0.245 | 20 | 4.9 | 12.2 | 94.93 | 0.532 |
| var7 | 260416-7 | | | | | | |
| ENC-001-1 | 360- | 0.136 | 20 | 2.72 | 6.8 | 96.96 | 0.346 |
| var8 | 260416-8 | | | | | | |
| ENC-001-1 | 360- | 0.098 | 20 | 1.96 | 4.9 | 95.78 | 0.536 |
| var9 | 270416-9 | | | | | | |
| ENC-001-1 | 360- | 0.149 | 20 | 2.98 | 7.4 | 96.24 | 0.378 |
| var10 | 270416- | | | | | | |
| ENC-001-1 | 360- | 0.145 | 20 | 2.9 | 7.2 | 97.8 | 0.889 |
| varl 1 | 270416- | | | | | | |
| ENC-001-1 | 360- | 0.06 | 20 | 1.2 | 3 | 97.52 | 1.93 |
| var12 | 270416- | | | | | | |
| ENC-001-1 | 360- | 0.046 | 20 | 0.92 | 2.3 | 97.28 | 0.123 |
| var13 | 270416- | | | | | | |
| ENC-001-1 | 360- | 0.061 | 20 | 1.22 | 3 | 97.75 | 0.351 |
| var14 | 270416- | | | | | | |
| ENC-001-1 | 360- | 0.085 | 20 | 1.7 | 4.2 | 96.35 | 0.28 |
| var15 | 270416- | | | | | | |

### Methods

### Gene Synthesis

Heavy and light chain genes were synthesised and sub-cloned into Lonza Biologics GS Xceed™ gene expression system vectors, pXC-17.4 and pXC-18.4. A Kozak sequence preceded the signal sequence, following the N-terminal restriction site.

### Single Vector Construction

Heavy Chain vectors were constructed by sub-cloning the heavy chain into the vector pXC-18.4, while light chain vectors were constructed by sub-cloning the light chain into the vector pXC-17.4 using the 5′ restriction site HindIII and the 3′ restriction site EcoRI. Restriction digests were electrophoresed on 0.7% agarose gels and the relevant fragments gel extracted using a QIAquick gel extraction kit (QIAGEN, 28704) according to manufacturer's instructions. Ligations were set-up at a final volume of 21 μl, and incubated at room temperature for 5 min. 10 μl aliquots of the ligation reaction were used to transform One Shot Top 10 Chemically Competent Escherichia coli cells (Life Technologies, C404003) using the heat-shock method according to manufacturer's instructions. Cells were spread onto ampicillin-containing (50 μg/ml) Luria Bertani agar plates (LB Agar, Sigma-Aldrich L7025) and incubated overnight at 37° C. until bacterial colonies were evident. The recombinant colonies were screened for the presence of the appropriate insert by colony PCR. Simultaneously, bacterial colonies were picked into 5 ml Luria Bertani (LB) medium (LB, Sigma-Aldrich L7275) containing 50 μg/ml ampicillin and incubated at 37° C. overnight with shaking. Vector DNA was isolated using the QIAGEN miniprep system (QIAprep spin miniprep kit, 27104) and eluted in 30 IA EB buffer. Insert presence was further corroborated by restriction mapping and agarose gel analysis (0.7%) using the following combinations of restriction endonucleases: EcoRI and HindIII, PvuI and NotI, EcoRI and BamHI. Final sequence identity was confirmed via sequencing.

### DNA Amplification

For Giga preps, single bacterial cultures were used to inoculate a starter culture which was subsequently used to inoculate 1.0 L PlasmidPlus media containing 50 μg ampicillin and incubated at 37° C. overnight with shaking. Vector DNA was isolated using the QIAGEN Gigaprep system (Qiagen, 12291). In all instances, DNA concentration was measured using a Nanodrop 1000 spectrophotometer (Thermo-Scientific) and adjusted to 1 mg/ml. DNA quality was assessed by measuring the absorbance ratio at 260 and 280 nm.

### Routine Culture of CHOKISV GS-KO Cells

CHOK1SV GS-KO cells were cultured in CD-CHO media (Life Technologies, 10743-029) supplemented with 6 mM L-glutamine (Life Technologies, 25030-123). Cells were incubated in a shaking incubator at 36.5° C., 5% CO2, 85% humidity, 140 rpm. Cells were routinely sub-cultured every 3-4 days, seeding at 0.2× 106 cells/ml and were propagated in order to have sufficient cells available for transfection. Cells were discarded by passage 20.

### Transient Transfection of CHOK1SV GS-KO Cells

Transient transfections were performed using CHOK1SV GS-KO cells which had been in culture a minimum two weeks. Cells were sub-cultured 24 h prior to transfection. All transfections were carried out via electroporation using the Gene Pulse XCell (Bio-Rad). For each transfection, viable cells were resuspended in pre-warmed CD-CHO media supplemented with 6 mM L-glutamine to 2.86×107 cells/ml. 40 μg of each heavy chain SGV DNA and each light chain SGV DNA (Table 10) was aliquoted into each cuvette (Bio-Rad, GenePulser cuvette, 0.4 cm gap, 165-2091) and 700 μl cell suspension added. Cells were electroporated at 300 V, 900 ρF. Transfected cells were transferred to pre-warmed media in Erlenmeyer flasks and the cuvette was then rinsed twice with pre-warmed media and the contents also transferred to the flasks. Transfected cultures were incubated in a shaking incubator at 36.5° C., 5% CO2, 85% humidity, 140 rpm for 6 days. Cell viability was measured at the time of harvest using a Cedex HiRes automated cell counter (Roche).

TABLE 10
Combinations of Heavy and Light chains used

| | | Light chains | | | | | |
|---|---|---|---|---|---|---|---|
| Combinations | | VL | VL1 | VL2 | VL3 | VL4 | VL5 |
| Heavy | VH | Clone 33 | | | | | |
| chains | VH1 | | 33_var1 | 33_var4 | 33_var7 | 33_var10 | 33_var13 |
| | VH2 | | 33_var2 | 33_var5 | 33_var8 | 33_var11 | 33_var14 |
| | VH3 | | 33_var3 | 33_var6 | 33_var9 | 33_var12 | 33_var15 |

### Primary Recovery

For small scale transient production, cultures were harvested by centrifugation at 2000 rpm for 10 min and filtered using a 0.22 gm PES membrane to obtain clarified supernatant.

### Protein A Affinity Chromatography

For the transient cultures, clarified supernatant was purified using a pre-packed 5 ml HiTrap MabSelect SuRE column (GE Healthcare, 11-0034-94) on an AKTA purifier (run at 10 ml/min).

In all cases, the column was equilibrated with 50 mM sodium phosphate, 125 mM sodium chloride, pH 7.0, washed with 50 mM sodium phosphate and 1 M sodium chloride pH 7.0 followed by re-introduction of equilibration prior to elution. The molecule was eluted with 10 mM sodium formate, pH 3.5. Eluted fractions were immediately pH adjusted by neutralizing with 2×PBS buffer, pH 7.4 and titrated to approximately pH 7.2 by the addition of dilute sodium hydroxide solution. Products were concentrated to >1 mg/ml for product analystics using an Amicon Ultra-15 Centrifugal Filter Unit with a 30 kDa MWCO (Merch, Millipore).

### SE-HPLC

Duplicate samples were analysed by SE-HPLC on an Aligent 12000 series HPLC system, using a Zorbax GF-250 9.4 mm IS×25 cm column (Agilent). 80 ul aliquots of 1 mg/ml samples (or stock concentrations if samples are <1 mg/ml) were injected and run in 50 mM sodium phosphate, 150 mM sodium chloride, 500 mM arginine, pH 6.0 at 1 ml/min for 15 minutes. Soluble aggregate levels were analysed using Chemstation software. Signals arising from buffer constituents were analysed by blank buffer injection and are omitted in the data analysis unless indicated otherwise.

### SDS-PAGE Analysis

Reduced samples were prepared for analysis by mixing with NuPage 4×LDS sample buffer (Life Technologies, NP0007) and NuPage 10× sample reducing agent (Life Technologies, NP0009), and incubated at 70° C., 10 min. For non-reduced samples, the reducing agent and heat incubation were omitted. Samples were electrophoresed on 1.5 mm NuPage 4-12% Bis-Tris Novex pre-cast gels (Life Technologies, NP0315/6) with NuPage MES SDS running buffer under denaturing conditions. A 10 uL aliquot of SeeBlue Plus 2 pre-stained molecular weight standard (Life technologies, LC 5925) and of a control antibody at 1 mg/ml were included on the gel. 1 ug of each sample was loaded onto the gel. One electrophoresed, gels were stained with InstantBlue (TropleRed, ISB01L) for 30 min at room temperature. Images of the stained gels were analysed on a BioSpectrum Imaging System (UVP).

### Endotoxin Measurements

Endotoxin levels of purified protein at 1 mg/ml concentration (unless indicated otherwise) were measured using the Endosafe-PTS instrument, a cartridge based method based on the LAL assay (Charles River). Due to the nature of the Products, in an effort to minimise interference of the products to the LAL measurement, a short heat inactivation step of 90° C. for 10 minutes was applied to denature the products prior to LAL measurement.

### Results

TABLE 11
Characteristics of antibody variants of clone 33

| | 33_VL | 33_VL_1 | 33_VL_2 | 33_VL_3 | 33_VL_4 | 33_VL_5 |
|---|---|---|---|---|---|---|
| | Variant | | | | | |
| | Aggregation score predicted | | | | | |
| | Yield | | | | | |
| | Titer | | | | | |
| | Monomer % | | | | | |
| | Aggregation % | | | | | |
| | Endotoxin (EU/mg) | | | | | |
| 33_VH | Clone 33 | | | | | |
| | 2.6 | | | | | |
| | 1.06 | | | | | |
| | 2.65 | | | | | |
| | 68.92 | | | | | |
| | 30.67 | | | | | |
| | 4.12 | | | | | |
| 33_VH_1 | | 33_var1 | 33_var4 | 33_var7 | 33_var10 | 33_var13 |
| | | 2.8 | 3.1 | 3.1 | 2.1 | −0.1 |
| | | 3.6 | 5.34 | 4.9 | 2.98 | 0.92 |
| | | 9 | 13.3 | 12.2 | 7.4 | 2.3 |
| | | 96.38 | 96.7 | 94.93 | 96.24 | 97.28 |
| | | 3.61 | 3.28 | 4.92 | 3.70 | 2.72 |
| | | 7.25 | 0.477 | 0.532 | 0.378 | 0.123 |
| 33_VH_2 | | 33_var2 | 33_var5 | 33_var8 | 33_var11 | 33_var14 |
| | | 1.2 | 1.6 | 1.6 | 0.5 | −1.7 |
| | | 2.05 | 1.62 | 2.72 | 2.9 | 1.22 |
| | | 5.1 | 4 | 6.8 | 7.2 | 3 |
| | | 97.08 | 98.5 | 96.96 | 97.8 | 97.75 |
| | | 2.92 | 1.50 | 3.04 | 2.19 | 2.22 |
| | | 0.430 | 0.489 | 0.346 | 0.889 | 0.351 |
| 33_VH_3 | | 33_var3 | 33_var6 | 33_var9 | 33_var12 | 33_var15 |
| | | 2.2 | 2.6 | 2.6 | 1.5 | −0.7 |
| | | 2.45 | 1.92 | 1.96 | 1.2 | 1.7 |
| | | 6.1 | 4.8 | 4.9 | 3 | 4.2 |
| | | 97.8 | 98.08 | 95.78 | 97.52 | 96.35 |
| | | 1.49 | 1.91 | 4.12 | 2.37 | 3.57 |
| | | 0.378 | 0.430 | 0.536 | 1.93 | 0.28 |

### Conclusion

Small scale transient transfections of the parental antibody ENC-001/1WT and 15 variants which has been re-engineered to reduce their propensity to aggregate (33_var1-33_var15) were established to express, purify and analyse aggregation profiles of the products (Table 11). Yields derived from the transient cultures varied between 2.3 mg/L at the lowest and to 13.3 mg/b at the highest end. The obtained yields for these transient cultures are summarized in Table 11 and show up to a 5-fold increase in the titre of the variants compared to the ENC-001/1_WT product. SDS-page analysis confirms the presence of the products at a high level of purity and are comparable to an in-house. IgG1 antibody control. Aggregate levels were determined by SE-HPLC. With the exception of clone33_WT, all variants exhibited high levels of monomer with no variant exhibiting less than 94.9% monomeric species. Fragments were present at a low level, with none of the variants exceeding levels of >0.2%.

Endotoxin levels were determined and initial measurements indicated interference of the LAL measurement by the product which is presumed to be due to the biological function of the molecules. In an effort to minimize this interference a heat denaturing step was included pre-measurement. Post heat-treatment, all of the variants showed a LAL measurement of <1 EU/mg, apart from WT, variant 1 and variant 12 which showed an elevated measurement of endotoxin.

### Example 3. ELISA Assay Clone 33E3 Variants

Various antibody clones are tested for binding to fibronectin-EDA using an ELISA assay. Binding of variants 1-15 to either the EDA domain or EDA domain peptide is studied. The EDA domain fragment is a purified recombinant HIS-tagged protein fragment that contains 90 amino acids of the EDA domain of Fibronectin.

The EDA domain peptide is a 29 amino acid peptide of which 27 are part of the EDA domain of Fibronectin. The peptide sequence (except two aa at the end) can be found back in the fragment sequence.

### Results

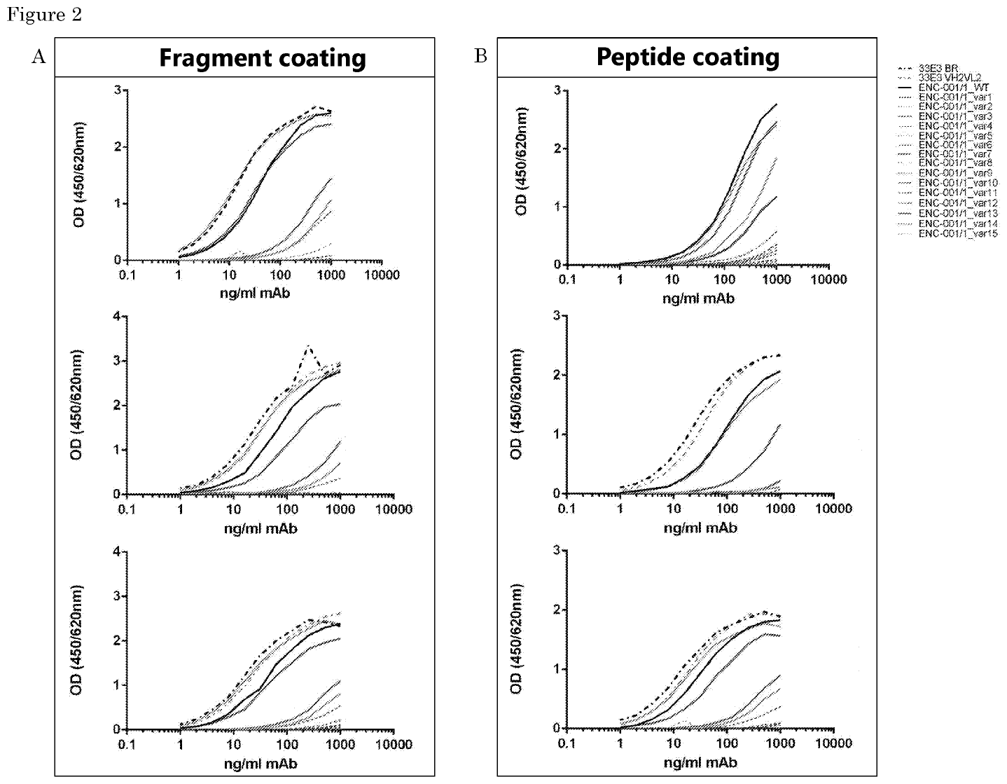

Variant 10 and clone 33 E3 wild type shows strong binding affinity to both the EDA domain of fibronectin fragment and peptide (FIGS. 2 a and b). Variant 13 shows strong binding affinity to the EDA domain of fibronectin fragment (FIG. 2a). Variant 7, 1 and 13 show intermediate binding affinity to the EDA domain of fibronectin peptide (FIG. 2b).

### Conclusion

The antibody clone33E3_Variant 10 has similar binding affinity for fibronectin-EDA as compared to Clone 33E3.

### Example 4. ELISA with EDA Peptide or Fragment as Coating

### Methods

1. Coat 96 wells plates with 50 μl/well EDA fragment (160 ng/ml) diluted in PBS for 1 h at 37° C.
2. Remove coating (flick off plate) and wash plate 3× (200 μl/well) with wash buffer at RT.
3. Add 200 μl/well block buffer and incubate 1 h at RT (shaking 100 rpm).
4. Remove block-buffer (flick off plate).
5. Apply 50 μl/well standard and incubate 1 h at RT (shaking 100 rpm).

| | | | |
|---|---|---|---|
| standards | ng/ml mAb | mAb solution | Block buffer |

| −1 | 1000 | ng/ml | 1 ul 1 mg/ml | | 999 ul |
|---|---|---|---|---|---|
| −2 | 500 | ng/ml | 120 | ul (1) | 120 ul |
| −3 | 250 | ng/ml | 120 | ul (2) | 120 ul |
| −4 | 125 | ng/ml | 120 | ul (3) | 120 ul |
| −5 | 62.5 | ng/ml | 120 | ul (4) | 120 ul |
| −6 | 31.3 | ng/ml | 120 | ul (5) | 120 ul |
| −7 | 15.6 | ng/ml | 120 | ul (6) | 120 ul |
| −8 | 7.8 | ng/ml | 120 | ul (7) | 120 ul |
| −9 | 3.9 | ng/ml | 120 | ul (8) | 120 ul |
| −10 | 2.0 | ng/ml | 120 | ul (9) | 120 ul |
| −11 | 1.0 | ng/ml | 120 | ul (10) | 120 ul |

| | | | |
|---|---|---|---|
| −12 | blank | X | 120 ul |

6. Wash plate 3× (200 μl/well) with wash buffer at RT.
7. Add 50 μl/well goat-a-human IgG HRPO (1:5000× diluted in block buffer) and incubate 1 h at RT (shaking 100 rpm).
8. Wash plate 3× (200 μl/well) with wash-buffer at RT.
9. Apply 50 μl/well TMB solution and incubate at RT.
10. Stop reaction with 50 μl/well Stop Reagent.
11. Measure OD 450 nm and reference at OD 620 nm using a microplate reader.

### Materials:

Wash buffer: PBS+0.05% Tween20
Block buffer: PBS+1% BSA
Stop solution: 0.16M sulfuric acid (1 ml stock in 116 ml aqua dest).

### Results and Conclusions

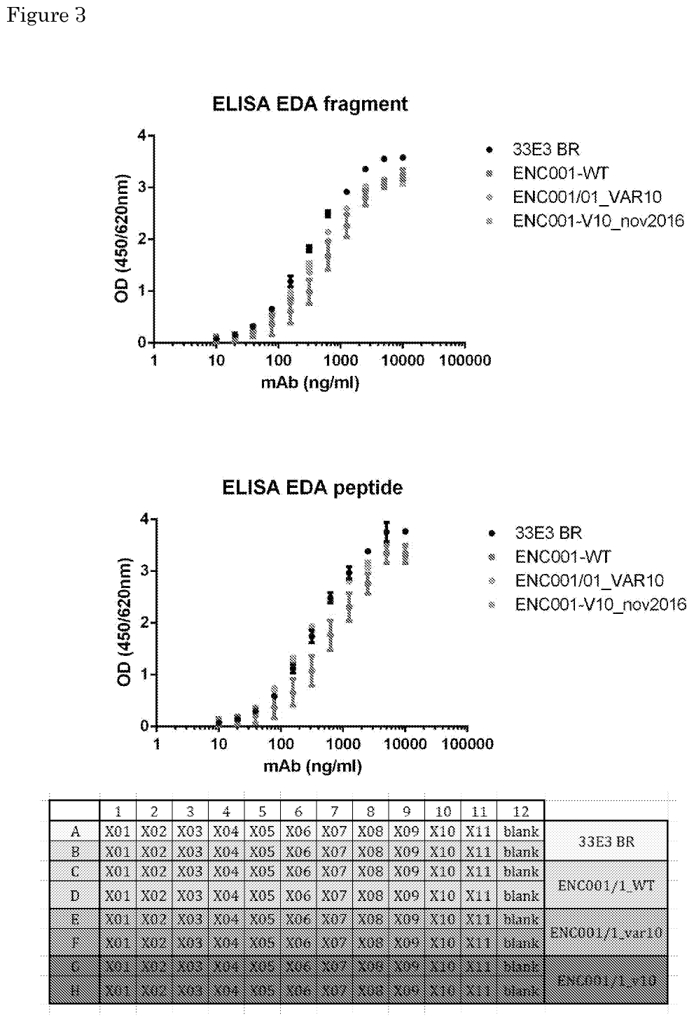

ELISA results are displayed in FIG. 3.

### Example 5. Adhesion Assay

### Cell Adhesion Assay

Ninety-six (96) wells plate was coated with 1 μM of EDA-his or III4-his fragments for 1 hour at 37° C., then blocked with PBS 1% BSA. NIH3T3 0.5×10\hat{ }5 cells were added per well in bare DMEM and incubated for 1 hour at 37° C. After washing of unattached cells, the attached cells were fixed and stained using 0.5% crystal violet 1% formaldehyde, 20% methanol. The plate was read with a microplate reader at 540 nm.

### Conclusion

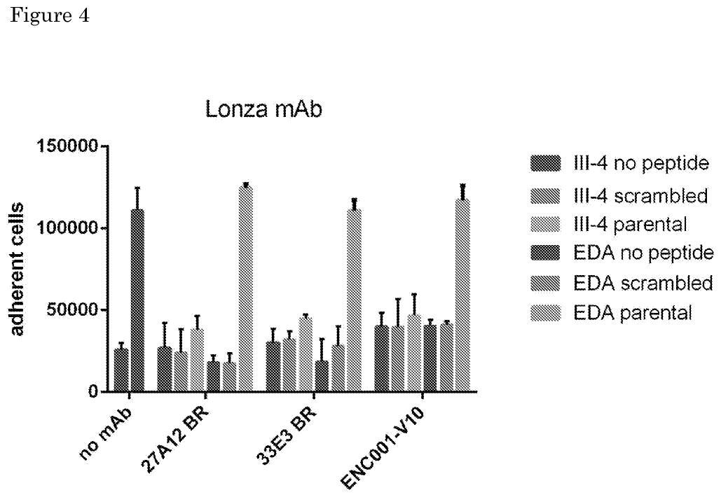

Variant 10 shows similar adhesion results compared to antibody clone 27A12 and parental clone 33E3 (FIG. 4).

### Example 6. Construction, Selection and Evaluation of Clonal GS-CHO Cell Lines Expressing the ENC001_v10 Antibody

Construction, selection and evaluation of a clonal GS-CHO cell line suitable for cGMP manufacture of the IgG4 antibody ENC001_v10 using Lonza's GS Xceed™ Expression System.

A vector, containing the heavy and light chain genes encoding the polypeptides for the antibody ENC00_v10, was constructed, based on sequences encoding the HC and LC polypeptides of the antibody ENC001_v10. DNA sequences encoding the HC and LC genes were generated and subsequently synthesised.

Three transfections were performed using the CHOK1SV GS-KO host cell line and the GS vector pENC001_v10/DGV to generate stable CHOK1SV GS-KO transfectant minipools expressing the ENC001_v10 antibody. The transfectant minipools were assessed for product expression and product concentrations of <1.2 to 180.6 mg/L were achieved. The highest producing minipools were combined to create enriched pools.

From 13440 wells, a total of 5743 wells were identified as containing growing cells. From these wells, 539 cell lines were screened for product expression; 301 colonies produced quantifiable levels of product. The 77 highest ranked cell lines were transferred to suspension culture.

Following adaptation to suspension culture, an assessment of the growth and productivity of the 48 highest ranked cell lines was undertaken. This assessment was performed in fed-batch miniature bioreactor cultures using materials and conditions that mimicked the fed-batch GS-CHO bioreactor culture process. The concentration of the ENC001_v10 antibody at harvest ranged from 2249 to 6435 mg/L, as determined by Protein A HPLC.

Eight cell lines were selected for further evaluation based upon high productivity in the fed-batch assessment, acceptable growth characteristics during routine subculture in shake-flask cultures and the parental pool from which the cell lines were derived. The images from the screening stage were also reviewed to support that the lead cell lines had emerged from a single colony.

The product produced by each of the 8 cell lines was comparable when analysed by SDS electrophoresis, ic-IEF, GP HPLC, and N-glycan UPLC-MS.

A research cell bank of each of the 8 selected lead candidate cell lines was cryopreserved.

Cell lines for selection and evaluation were constructed by transfecting CHOK1SV GS-KO host cells with the DGV. The CHOK1SV GS-KO host cell line is a derivative of the CHOK1SV host cell line with the endogenous gene for GS ‘knocked out’.

### Materials and Methods

### Cell Culture

Revival from Cryopreservation

Cells were revived from vials of cryopreserved stocks by rapidly warming to 37.0° C. and diluting into ˜50 mL of growth medium. The DMSO was removed by centrifuging the cells, discarding the supernatant and resuspending the cells in fresh growth medium. Cultures were seeded at 0.3×106 viable cells/mL after recovery from cryopreservation and initially subcultured on Day 3. Thereafter, cultures were seeded at 0.2×106 viable cells/mL and subcultured every 4 days.

### Static Cell Culture

For static culture, the containers used were 96-WPs. These cultures were incubated at 35.5 to 37.0° C. in a humidified atmosphere of 10% v/v CO2 in air.

### Culture in 96-Deep Well Plates

Cultures of cell lines in 96-DWPs were incubated on a shaking platform at a sufficient rotational speed to maintain the cells in suspension, and at 35.5 to 37.0° C. in a humidified atmosphere of 5% v/v CO2 in air. After the initial transfer into shaken 96-DWPs, cell lines were maintained on a 4 day subculture regime.

### Generation of Stable CHOK1SV GS-KO Transfectant Pools

### Transfection to Generate Transfectant Minipools

The CHOK1SV GS-KO host cell line was revived from a vial of cryopreserved WCB into an appropriate CDACF growth medium (supplemented with L-glutamine) in suspension culture. The CHOK1SV GS-KO host cell line was serially subcultured in this medium on a 4 day subculture regime. The culture volume was expanded at each subculture until sufficient cells were available to undertake the required number of transfections.

The CHOK1SV GS-KO host cell line was prepared for transfection by centrifuging and resuspending in same growth medium (but without L-glutamine supplement) at an appropriate VCC.

For each transfection, ˜0.8 mL of cell suspension and linearised plasmid DNA were added to a single electroporation cuvette. The electroporation cuvette was then placed in the electroporation apparatus (Gene Pulser Xcell™, Bio-Rad) and a single pulse of 300 V, 900 μF was delivered.

Following each transfection, the cells from the cuvette were diluted into an appropriate volume of the growth medium (without MSX) and distributed across 96-WPs. The plates were incubated. The day after transfection, an appropriate volume of the selective medium (fresh growth medium supplemented with an appropriate concentration of MSX) was added to each well of the 96-WPs. The culture in a single well of the 96-WP is termed a ‘minipool’.

### Expansion of Transfectant Minipools

After an appropriate incubation period in static culture, the medium in the transfectant minipool cultures was replaced with fresh selective medium. The confluence of each well was subsequently determined at appropriate intervals until the majority of the wells exhibited suitable cell growth. At this point, the transfectant minipools were transferred into suspension culture. The contents of each well were subcultured into corresponding wells on 96-DWPs, using an appropriate growth medium supplemented with MSX. The minipools in 96-DWPs were then maintained as suspension cultures by incubating the plates on a shaking platform.

### Productivity Assessment of Transfectant Minipools and Generation of Enriched Transfectant Pools

After an appropriate incubation period in suspension culture, supernatant samples were aseptically removed from wells in 96-DWPs and transferred to Octet® System compatible 96-WPs. The sampled CCSs were subsequently assayed for product concentration using an Octet® System. The first 2 columns from each plate (16 wells) were not sampled to make room for standards, blanks and IACs on the Octet® plates. For each 96-DWP, minipools were ranked according to product concentration data and the rank positions were used to select minipools to generate enriched transfectant pools. For 6 of these enriched pools, the culture contents of the highest ranked minipools from each of 6 transfectant 96-DWPs were combined in an intermediate container to generate 6 enriched transfectant pools. For the final enriched pool, the highest ranked minipools across two 96-DWPs were combined.

The VCC of each enriched transfectant pool was determined by Vi-CELL XR® and a shake-flask culture of each enriched transfectant pool was prepared using an appropriate growth medium supplemented with MSX. These enriched transfectant pool cultures were used for single cell sorting using a FACS.

### Cell Cloning

Clonal cell lines were generated from the enriched transfectant pools using a FACS. The FACS Aria™ III (Becton Dickinson) was prepared for aseptic, single-cell sorting as detailed in the manufacturer's guidelines. The sample lines were replaced and the fluidics system sanitised before the start of the first day of sorting.

### FACS Instrument Settings

The instrument settings were set at the beginning of each sort day. Gating criteria were established on dual parameter dot plots to select populations of single cells. Plots of FSC and SSC emission data were used to identify single viable cells for sorting. The instrument set-up and position of the gates were verified as suitable for single-cell sorting. This was achieved by initially sorting fluorescent beads and then ER-Tracker™ Green (BODIPY® FL glibenclamide: Life Technologies) stained cells onto 96-WP lids using the markings on the lid, which correspond to the wells in the plate base, as the targets.

The targets (on 96-WP lids) were checked manually using a fluorescence microscope and the number of particles in the target was recorded. The expectation is that when sorting fluorescent beads, all 96 targets will contain a single bead. If this was not achieved, the set-up of the instrument was repeated. When sorting cells, the proportion of targets that contain a single cell is not always unity because cells are not perfect spheres. Therefore, where more than 1 target of the 96 targets sorted with stained cells was seen to contain 2 or more cells, the set-up of the instrument was repeated. The exercise using stained cells was repeated at the end of each sort session to confirm that the set-up of the instrument and the gating positions for single-cell sorting were robust. No reagents were added to aid identification and selection of cells for sorting. The probability of monoclonality was calculated for each session using the results from the manually checked targets at the beginning and end of each session.

### Single Cell Sorting

Enriched transfectant pools, prepared in an appropriate growth medium (without MSX) supplemented with SP2 just before sorting, were single-cell sorted based on gating criteria established. Unstained cells were sorted at 1 cell/well into 96-WPs containing an appropriate cloning medium.

### Colony Screening

The ICCMS is an automated system that is used to capture and analyse digital images of all the wells of 96-WPs derived from single cell sorting. Image-based analysis was used to identify wells containing single colonies and to estimate their confluence. At appropriate intervals after single cell sorting, cloning plates were screened using the ICCMS to identify wells containing colonies.

Images captured by the ICCMS demonstrating the growth of individual colonies across the screens were retrospectively examined for each of the cell lines selected for consideration as lead cell lines. Colonies had to show acceptable traceability, formation and maintenance of a single colony throughout the screens to be considered as a candidate for the lead cell line.

### Transfer to Suspension Culture in 96-DWPs

Static cultures of selected cell lines were manually transferred into 96-DWPs for subsequent suspension culture in GS-CHO subculture medium. After the initial transfer to 96-DWPs, cultures of cell lines in 96-DWPs were grown in suspension mode and subcultured on a 4 day regime. Cell lines were maintained in this medium on a 4 day subculture regime thereafter until expansion of selected cell lines to shake-flask cultures.

Expansion of Suspension Cultures from 96-DWP

Suspension cultures of cell lines were expanded from the 96-DWPs, via a shaken intermediate container, to shake-flasks, in the appropriate subculture medium. The VCC in the 96-DWP, prior to transfer into the intermediate container, was determined using a Celigo®. These intermediate cultures were incubated: on Day 4 (day of subculture) their VCCs were determined using a Vi-CELL XR®. Cultures of the selected cell lines were subsequently serially subcultured, in the same medium, on a 4 day subculture regime: culture volumes were expanded, where required, using appropriately sized containers.

### Corrected Culture Viability

The corrected culture viability at harvest of the FMB cultures was also calculated. This is the ratio of the VCC on day of harvest to the maximum total cell concentration recorded over the culture multiplied by 100. This is to account for both intact dead cells and also those that have lysed during the culture and thus not measurable in the harvest samples.

### Protein A HPLC

Product concentrations in CCS samples were quantified by Protein A affinity H PLC. Product was selectively bound to a POROS Protein A immunodetection cartridge. Non-bound material was washed from the column and bound product released by changing solvent conditions. The absorbance of the eluate was monitored at A280 nm. Eluted product was quantified against an appropriate standard and corrected using a product specific extinction coefficient of 1.41 (ε 0.1%, 1 cm).

### MabSelect SuRe Protein A Affinity Purification

Product purification, for analytical purposes only, was performed using custom packed MabSelect SuRe Protein A Sepharose columns (160 μL: Phynexus Inc.) with pre-set binding and elution conditions. Column loads were applied using a Micro Extraction (automated) instrument (Phynexus Inc.). Antibody was reversibly bound to the MabSelect SuRe Protein A matrix. Non-bound material was washed from the column and bound product molecules released by changing solvent conditions. Absorbance at 280 nm with a product specific extinction coefficient of 0.1% ε1 cm=1.41 was used to measure the product concentration in the MabSelect SuRe Protein A eluates.

### Oligosaccharide Analysis by UPLC High Throughput N-Glycan Analysis

The UPLC-MS high throughput N-glycan analysis platform comprises of high throughput glycan preparation using a GlykoPrep Rapid 2-AB kit with the AssayMAP Bravo Liquid Handler followed by UPLC and mass spectrometry analysis. The sample preparation workflow consisted of an automated purification and normalization step followed by the N-glycans release by digestion with the enzyme peptide-N glycosidase F, separation from the glycoproteins, fluorescent labelling with the fluorophore 2-aminobenzamide and clean-up for analysis. The labelled glycans were analysed by hydrophilic interaction UPLC coupled with electrospray time of flight mass spectrometry. Analysis was performed using an AQUITY UPLC H-Class Bio system and an AQUITY UPLC fluorescence detector in-line with a Xevo G2S Q-TOF system operated in sensitivity mode and positive ionization mode.

The quantitation and identification of oligosaccharide structures was performed using the Glycan Workflow in UNIFI 1.7 software. A 2-aminobenzamide-labelled dextran ladder was used to calibrate and normalize 2-aminobenzamide-labelled glycan retention time into glucose units. Initial assignment of oligosaccharide species for neutral and charged oligosaccharide profiling was made based on comparison of glucose unit to the NIBRT Glycan database. Those initial assignments were confirmed by mass analysis. The percentage of each glycan was based on the area of each peak relative to the total integrated peak area.

### Aggregate Analysis by GP HPLC

GP HPLC was used to separate product monomer from both aggregates and fragments. The monomeric component was identified by its characteristic retention time and position relative to calibration markers. Aggregate analysis was performed using a TSK G3000 column (Hichrom). Product components were detected by A280 nm measurements and peak chromatograms were analysed using Empower™ software (Empower™ Software Solutions). The proportion of sample components was determined by calculation of the peak areas of each component relative to the total integrated peak area.

### Results and Discussion

### Transfection of CHOK1SV GS-KO Host Cells to Generate Stable GS-CHO

### Transfectant Minipools

CHOK1SV GS-KO host cells were revived from cryopreserved WCB into suspension culture in the medium CD CHO/6 mM L-glutamine and culture volumes expanded. Subsequently, host cells on Day 3 of subculture, at generation 53.5 (6.5 generations beyond that of the WCB 760-W3), were stably transfected with the GS vector pENC001_v10/DGV. Three electroporations (1, 2 and 3) were performed, generating 864 stable GS-CHO transfectant minipools distributed between 9×96 WPs (Plates 1A, 1B, 1C, 2A, 2B, 2C, 3A, 3B and 3C). The day after transfection, the selective medium CM119 supplemented with 100 μM MSX was added to each well of the 96-WPs to give a final concentration of 50 μM MSX.

After an appropriate incubation time, the medium in the transfectant minipool cultures was replaced with fresh selective medium. Plates 1A to 3C were re-fed with the selective medium CM119 supplemented with 50 μM MSX.

### Generation and Selection of Enriched Transfectant Pools

After adequate cell growth in the static 96-WP cultures of transfectant minipools was achieved, all 9 plates were expanded to suspension cultures in shaken 96-DWP in the medium CM119/50. Three days after transfer to suspension culture, 8 of the 96-DWPs were sampled to screen for protein production. Plate 3B was not progressed as low cell growth was observed.

Samples from 640 wells were assayed for protein production by the Octet® method and the product concentration data generated were used to identify the highest producing transfectant minipools. To minimise inter-plate and inter-assay differences, the ranking was performed independently for each assay plate. Overall product concentrations ranged between <1.2 to 180.6 mg/L. The transfectant minipools were subsequently expanded to suspension cultures in shaken 96-DWPs in the medium CM119 supplemented with 50 μM MSX.

Cultures of the 10 highest producing transfectant minipools on each plate, from 1A to 2C, were pooled together to generate 6 enriched transfectant pools (Pools 1 to 6). Pool 7 was generated by pooling the 10 highest producing minipools across plates 3A and 3C. The pooled cultures were maintained in CD CHO/SP4/50 μM MSX on a 3 day subculture regime before single cell sorting.

Isolation of Clonal Cell Lines from Enriched Transfectant Pools Using a FACS

Clonal cell lines were obtained from the 7 enriched transfectant pools using a FACS operated in single-cell deposition mode. Cells for sorting were identified using their FSC and SSC emissions only and were not stained or labelled.

Cells were sorted at 1 cell/well into 96-WPs containing the CDACF growth medium CM104. The cells were sorted in 8 sort sessions over 2 days. Each pool was sorted in one successful sort session except pool 3 where the first session, identified as session 3, was stopped prematurely due to the stream becoming unstable. All 5 plates sorted during session 3 were discarded. In total, 13440 wells were targeted to be seeded with a single cell.

To estimate the probability of monoclonality, 960 targets were microscopically examined: 958 contained a single cell, whilst 2 contained 2 cells. The probability of monoclonality was estimated at the start and end of each of the 8 sort sessions undertaken in this programme.

The estimates for the probability of monoclonality for all sort sessions were above the acceptable limit of 0.990. Overall, the probability of monoclonality at the beginning and end of each successful sort session was high and any difference was less than 0.010. Thus, the behaviour of the FACS instrument between the beginning and end of all 7 successful sort sessions was considered to be similar. The cell lines generated in this programme can, with high probability, be considered monoclonal.

### Assessment of Colony Formation and Productivity in Static Culture

At approximately 1, 2 and 3 weeks after single cell sorting, the remaining 96-WPs were screened, to identify wells containing colonies. From ˜13440 wells sorted at 1 cell/well, 5743 wells in total were identified at Week 3 as containing colonies. The overall cloning efficiency was 42.7%. In total, 539 of these colonies were randomly selected for further evaluation.

A Hamilton STAR liquid handling workstation, integrated with a Cytomat™ automated incubator, was used to sample culture supernatants from 539 wells identified as containing single colonies. These samples were assayed for product concentration. Culture supernatants from the 539 clonal cell lines screened demonstrated a range of product concentrations. Quantifiable levels of product concentrations ranged from 1.2 to 61.0 mg/L for 301 out of the 539 colonies assessed. The remaining 238 colonies were below the LOQ (1.2 mg/L) of the assay.

Of the 301 clonal cell lines producing quantifiable levels of product, 77 cell lines with high ranked product concentrations were selected and transferred to suspension culture using the subculture medium CM66 and shaken 96-DWPs. The name subsequently referred to for each cell line was derived from its position when ranked by specific production rate. Selection was based on both ranking by productivity and parental pool, to maintain a high level of diversity. Suspension cultures were subcultured on a fixed 4 day regime. All subcultures in 96-DWP were based upon VCCs, determined using the Celigo® imaging system.

### Expansion of Suspension Cultures of Selected Cell Lines

All of the 77 cell lines were successfully adapted to suspension culture. Suspension cultures of 55 highest ranked cell lines from the productivity assessment were expanded from 96-DWPs to 125 mL shake-flask cultures. Suspension cultures of the 55 cell lines were serially subcultured in CM66. From the 55 cell lines transferred to suspension culture, 48 were selected for further evaluation based on acceptable growth and viability at routine subculture. Growth characteristics were considered acceptable if the VCC on day of subculture was consistently above 1.0×106 cells/mL and the culture viability above 90%.

### Cell Line Productivity Screening in FMB Culture

Suspension cultures of the 48 selected cell lines were then evaluated in FMB cultures. The purpose of the FMB screening step was to determine which cell lines would respond well, with respect to productivity, to the media and feeds used in GS-CHO bioreactor culture process (Version 8.6).

On Day 10 of FMB culture, the dissolved oxygen level was observed to be 23% in the vessel containing cell line ENC37, indicating a blockage of the filter. The filter was replaced and the further growth and viability of the cell line was unaffected. Later on, filter blockages also occurred on days 12 and 14 in two other vessels. In both cases, the dissolved oxygen level was found to have been at 2% for approximately 12 hours. Despite the filter being replaced, the further growth and viability of the cell lines, ENC24 and ENC17, were negatively impacted.

The product concentration at harvest ranged from 2249 to 6435 mg/L. The majority of the cell lines assessed in FMB culture were considered suitable for further evaluation.

### Selection of Lead Candidate Cell Lines for Further Evaluation

From the 48 selected cell lines assessed, 8 cell lines were subsequently selected for progression and cryopreservation of the RCB: ENC02, ENC05, ENC06, ENC15, ENC22, ENC28, ENC34 and ENC45.

The selection of the lead cell lines was based on the specific production rate, product concentration at harvest and lactate accumulation at harvest (data not shown) achieved in the FMB evaluation; the acceptable growth of the cell lines (consistently above 1.0×106 viable cells/mL at subculture); the evidence that each cell line arose from a single colony (by ICCMS screening) and the parental enriched transfectant pool of each cell line.

### Cryopreservation of RCBs

An RCB was cryopreserved for each of the 8 lead candidate cell lines.

A vial of the RCB for each cell line was recovered from cryopreservation to ensure that each cell line demonstrated acceptable growth characteristics after cryopreservation. Each cell line exhibited acceptable growth and viability upon revival (data not shown).

### Derivation of Lead Candidate Cell Lines

Characterisation of the ENC001_v10 Antibody from the FMB Cultures

Product from harvest supernatants from the FMB cultures of the 8 lead candidate cell lines was partially purified by MabSelect SuRe Protein A affinity chromatography before characterisation by non-reduced and reduced SDS electrophoresis, ic-IEF, oligosaccharide analysis and GP HPLC aggregate analysis.

Non-reduced and reduced samples of the Protein A purified ENC001_v10 antibody from the 8 FMB cultures were analysed by SDS electrophoresis, using a LabChip® GXII Protein instrument, for estimation of the molecular weights of the intact IgG and the LC and HC subunits, along with detection of any size variants if present. The system peaks were detected and the IAC profiles were as expected, therefore the assay results are considered to be valid. The electropherograms of the 8 reduced test samples each exhibited peaks with molecular weights corresponding to the expected LC molecular weight of approximately 25 kDa (28.0 to 28.3 kDa) and HC molecular weight of approximately 55 kDa (59.6 to 60.1 kDa). The electropherograms of all 8 non-reduced test samples exhibited a primary peak with a molecular weight corresponding to the expected intact IgG molecular weight of approximately 160 kDa (160.0 to 163.5 kDa).

Protein A purified ENC001_v10 antibody samples from the 8 FMB cultures were analysed by GP HPLC to determine levels of aggregate present. The GP HPLC chromatogram profiles of the 8 test samples were all comparable. The percentage of aggregate detected in all the test samples is ranged from 2.7 to 4.5%. Aggregate levels of <10% are considered acceptable. The level of aggregate after Protein A affinity chromatography determines the purification strategy, which can impact the purification yield and efficiency.

In summary, Protein A purified ENC001_v10 antibody samples from the FMB cultures of the 8 lead candidate cell lines assessed were shown to be similar to each other, when analysed by reduced and non-reduced SDS electrophoresis, ic-IEF, GP HPLC and N-glycan UPLC-MS. Any differences observed were considered to be minor and not sufficient to exclude any of these 8 cell lines from further evaluation. The product characteristics of these ENC001_v10 antibody samples were consistent with an IgG4 produced by a GS-CHO cell line.

### Example 7. ELISA EDA Fragment

Perform ELISA with EDA fragment to test epitope recognition from new clones of antibodies derived from original clone 33E3.10. Use samples mu33E3.10 clone 497 BR and ENC-001/01_var10 as positive controls.

### Methods

1. Coat 96 wells plates with 50 μl/well EDA fragment (EDA Livingstone monomer) (160 ng/ml) diluted in PBS for 1 hour at 37° C.
2. Remove coating (flick off plate) and wash plate 3× (200 μl/well) with wash buffer at RT.
3. Add 200 μl/well block buffer and incubate 1 h at RT (shaking 100 rpm).
4. Remove block-buffer (flick off plate).
5. Apply 50 μl/well samples and incubate 1 hour at RT (shaking 100 rpm) dilution in block buffer.

| | | | |
|---|---|---|---|
| Samples | ng/ml mAb | mAb solution | Block buffer |

| 1 | 1000 | ng/ml | 1 ul 1 mg/ml | | 999 ul |
|---|---|---|---|---|---|
| 2 | 500 | ng/ml | 120 | ul (1) | 120 ul |
| 3 | 250 | ng/ml | 120 | ul (2) | 120 ul |
| 4 | 125 | ng/ml | 120 | ul (3) | 120 ul |
| 5 | 62.5 | ng/ml | 120 | ul (4) | 120 ul |
| 6 | 31.3 | ng/ml | 120 | ul (5) | 120 ul |
| 1 | 15.6 | ng/ml | 120 | ul (6) | 120 ul |
| 8 | 7.8 | ng/ml | 120 | ul (7) | 120 ul |
| 9 | 3.9 | ng/ml | 120 | ul (8) | 120 ul |
| 10 | 2.0 | ng/ml | 120 | ul (9) | 120 ul |
| 11 | 1.0 | ng/ml | 120 | ul (10) | 120 ul |

| | | | |
|---|---|---|---|
| 12 | blank | X | 120 ul |

6. Wash plate 3× (200 μl/well) with wash buffer at RT.
7. Add 50 μl/well goat-a-human IgG HRPO (1:5000× diluted in block buffer) and incubate 1 h at RT (shaking 100 rpm).
8. Wash plate 3× (200 μl/well) with wash-buffer at RT.
9. Apply 50 μl/well TMB solution and incubate at RT for 5-10 minutes.
10. Stop reaction with 50 μl/well Stop Solution.
11. Measure OD 450 nm and reference at OD 620 nm using a microplate reader (use Biorad AND Multiskan to compare read-outs).

### Buffers

Wash buffer: PBS+0.05% Tween20
Block buffer: PBS+1% BSA
Stop Solution: 0.16 M Sulfuric acid

### Results

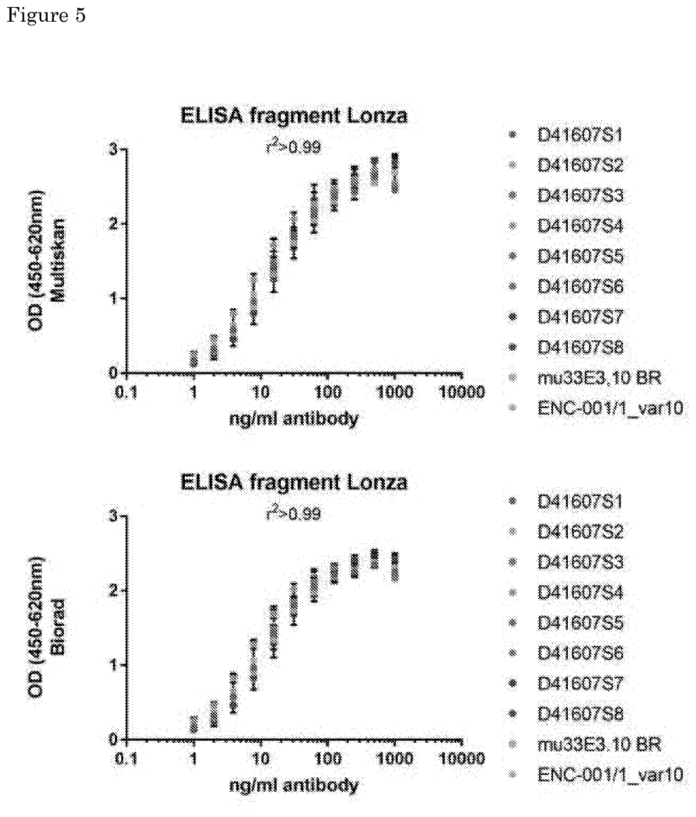

All clones show equal curves to positive controls mu33E3.10 clone 497 BR and ENC-001/01_var10 (FIG. 5).

### Conclusion

All clones recognize the EDA fragment.

### Example 8. Adhesion Assay

Test functionality from new batch of different clones all expressing variant 10 of antibodies in adhesion assay. Use samples mu33E3.10 clone 497 BR and ENC-001/1_var10 as positive controls.

### Method

1. Coat 96-well non-tissue culture coated flat bottom clear plates with 50 μl of the desired protein fragment in 1 μM, diluted with PBS.
2. Incubate for 1 hr at 37° C. Cover the plate with a plate seal.
3. Wash the plate 3× with PBS.
4. Block with 200 μl of 1% PBSA for 1 hour at RT while shaking at 100 rpm. Start with step 5 immediately after starting step 4:
5. Prepare antibody-peptide mixture
6. Preincubation of antibody with human or scrambled peptide: add 3.5 μl parental or scrambled peptide (2 mg/ml) to 350 μl mAb mixture and incubate 1 hour at RT while shaking at 100 rpm.
7. Remove 1% PBSA buffer from wells.
8. Wash the plate 3× with PBS.
9. Incubate wells with 50 μl of antibody-peptide mixture for 1 hour at RT while shaking at 100 rpm.
10. Label cells with Calcein AM during step 9 (cells are P12).
Detach cells using Trypsin-EDTA and resuspend in serum-free DMEM.
Centrifuge cell suspension at 300 g for 5 minutes and remove medium.
Resuspend 50 μg Calcein AM in 50 μl DMSO to get a 1 mM solution.
Resuspend cells in 50 ml DMEM and add 100 μl 1 mM Calcein (final [Calcein]=2 μM).
Incubate for 10 minutes in a 5% CO2 incubator.
Centrifuge cell suspension at 300 g for 5 minutes and remove medium.
Wash cells with 25 ml serum-free medium to remove free label.
Centrifuge cell suspension at 300 g for 5 minutes and remove medium.
Resuspend cells in serum-free DMEM to 2.0*106 cells/ml.
   11. Wash the plate 3× with PBS.
   12. To each coated well, add 100 μl of cell suspension (˜200.000 cells/well). Also add cells to column 1 (row B-G) to see how many cells bind to untreated wells. Keep remaining cells and store them in 5% CO2 incubator until the dilution series in step 16.
   13. Centrifuge the plates (top side up) at 10 g for 1 minute in order to reduce the variability inherent in settling of cells at 1×g onto the plate surface.
   14. Incubate for 1 hr at 37° C. in 5% CO2 incubator.
   15. Remove the non-adherent cells by centrifugation (top side down) at 50 g for 1 minutes. Drain media onto a stack of paper towels. Spin again (top side down) at 50 g for 1 min to get residual fluid off and drain on paper towels.
   16. Add a 2× dilution series of Calcein labeled cells (left-over cells from step 12) to column 12.
   17. Quantify Calcein labeled cells in the Spectramax M2E (filters: excitation=485 and emission=538) with software program Softmax pro 5.4.5.

### RESULTS AND CONCLUSION

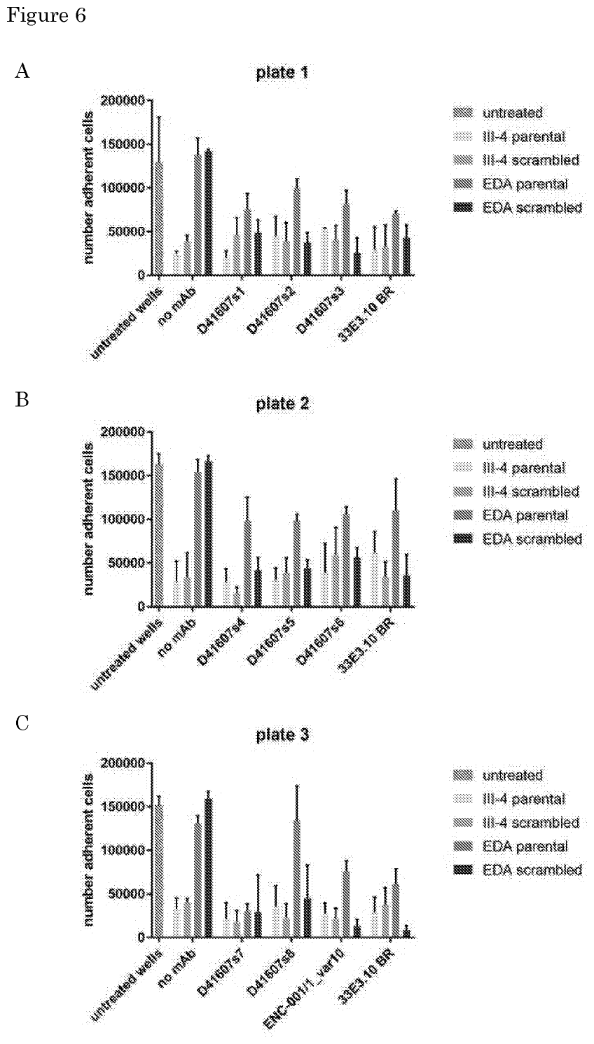

There is strong cell adhesion in EDA coated wells without antibody which is almost similar to untreated wells (column 1: no coating, no blocking) (FIG. 6).

Cell adhesion is blocked in presence of mAb pre-incubated with scrambled peptide for all antibodies; pre-incubation of mAb with parental peptide blocks mAb function and should allow cells to adhere to EDA.

SEQUENCES:
Bold underline indicates CDR regions
SEQ ID 1: amino acid sequences of the heavy chain variable region
(clone 33_VH1)
EVQLVESGGGLVQPGGSLRLSCAASGFTFSSSAMTWVRQAPGKGLEWVASISGGGTTYYADSVKGR
FTISRDNSKNTLYLQMNSLRAEDTAVYYCARSHYWGQGTLVTVSS
SEQ ID 2: amino acid sequences of a light chain variable region
(clone 33_VL4)
DIQMTQSPSSLSASVGDRVTITCRASQNVVTNVAWYQQKPGKAPKALIYSASYLYSGVPSRFSGSG
SGTDFTLTISSLQPEDFATYYCQQYSSYPYTFGGGTKVEIKR
SEQ ID 3: amino acid sequences of a light chain variable region
(clone 33_VL5)
DIQMTQSPSSLSASVGDRVTITCRASQGVVTSVAWYQQKPGKAPKALTYSASYLYSGVPSRFSGSG
SGTDFTLTISSLQPEDFATYYCQQYDSYPYTFGGGTKVEIKR
SEQ ID 4: amino acid sequence of the heavy chain of clone 33 (33_VH)
MELGLSWIFLLAILKGVQCEVQLVESGGGLVQPGGSLRLSCAASGFTFSNSAMTWVRQAPGKRLEW
VASISGGGTTYYPDSVKGRFTISRDNSKNTLYLQMNSLRAEDTAVYYCARSHYWGQGTLVTVSSAS
TKGPSVFPLAPCSRSTSESTAALGOLVKDYFPEPVTVSWNSGALTSGVHTFPAVLQSSGLYSLSSV
VTVPSSSLGTKTYTCNVDHKPSNTKVDKRVESKYGPPCPPCPAPEAAGGPSVFLFPPKPKDTLMIS
RTPEVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHYTQKSLSLSLGK
SEQ ID 5: amino acid sequence of the heavy chain 1 of clone 33 (33_VH1)
MELGLSWIFLLAILKGVQCEVQLVESGGGLVQPGGSLRLSCAASGFTFSSSAMTWVRQAPGKGLEW
VASISGGGTTYYADSVKGRFTISRDNSKNTLYLQMNSLRAEDTAVYYCARSHYWGQGTLVTVSSAS
TKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEAAGGPSVFLFPPKPKDTLMIS
RTPEVICVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHYTQKSLSLSLGK
SEQ ID 6: amino acid sequence of the heavy chain 2 of clone 33 (33_VH2)
MELGLSWIFLLAILKGVQCEVQLVESGGGLVQPGGSLRLSCAASGFTFSNSAMNWVRQAPGKGLEW
VASISGGGTTYYPDSVKGRFTISRDNSKNTLYLQMNSLRAEDTAVYYCARSHYWGQGTLVTVSSAS
TKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEAAGGPSVFLFPPKPKDTLMIS
RTPEVICVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHYTQKSLSLSLGK
SEQ ID 7: amino acid sequence of the heavy chain 3 of clone 33 (33_VH3)
MELGLSWIFLLAILKGVQCEVQLVESGGGLVQPGGSLRLSCAASGFTFSSSAMSWVRQAPGKGLEW
VATISGGGTTYYADSVKGRFTISRDNSKNTLYLQMNSLRAEDTAVYYCARSHYWGQGTLVTVSSAS
TKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEAAGGPSVFLFPPKPKDTLMIS
RTPEVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHYTQKSLSLSLGK
SEQ ID 8: amino acid sequence of the light chain WT of clone 33 (33_VL)
MDMRVPAQLLGLLLLWFPGARCDIQMTQSPSSVSASVGDRVTITCKASQNVVINVAWYQQKPGKAP
KALIYSASYRYSGVPSRFSGSGSGTDFTLTISSLQPEDFATYYCQQYNSYPYTFGGGTKVEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID 9: amino acid sequence of the light chain 1 of clone 33 (33_VL1)
MDMRVPAQLLGLLLLWFPGARCDIQMTQSPSSLSASVGDRVTITCRASQGVVINVAWYQQKPGKAP
KLLIYSASYRYSGVPSRFSGSGSGTDFTLTISSLQPEDFATYFCQQYNSYPYTFGGGTKVEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID 10: amino acid sequence of the light chain 2 of clone 33 (33_VL2)
MDMRVPAQLLGLLLLWFPGARCDIQMTQSPSSLSASVGDRVTITCRASQNVVTNVAWYQQKPGKAP
KALIYSASYRQSGVPSRFSGSGSGTDFTLTISSLQPEDFATYFCQQYNSYPYTFGGGTKVEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID 10: amino acid sequence of the light chain 3 of clone 33 (33_VL3)
MDMRVPAQLLGLLLLWFPGARCDIQMTQSPSSLSASVGDRVTITCRASQGVVINVAWYQQKPGKAP
KLLIYSASYRQSGVPSRFSGSGSGTDFTLTISSLQPEDFATYFCQQYSSYPYTFGGGTKVEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID 10: amino acid sequence of the light chain 4 of clone 33 (33_VL4)
MDMRVPAQLLGLLLLWFPGARCDIQMTQSPSSLSASVGDRVTITCRASQNVVTNVAWYQQKPGKAP
KALIYSASYLYSGVPSRFSGSGSGTDFTLTISSLQPEDFATYYCQQYSSYPYTFGGGTKVEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID 10: amino acid sequence of the light chain 5 of clone 33 (33_VL5)
MDMRVPAQLLGLLLLWFPGARCDIQMTQSPSSLSASVGDRVTITCRASQGVVTSVAWYQQKPGKAP
KALIYSASYLYSGVPSRFSGSGSGTDFTLTISSLQPEDFATYYCQQYDSYPYTFGGGTKVEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

1. An anti-fibronectin-EDA antibody or antigen binding fragment thereof comprising a heavy chain variable region and a light chain variable region,
wherein the heavy chain variable region comprises
a CDR1 having the sequence GFTFSSS or GFTFSNS;
a CDR2 having the sequence SGGGTTY; and
a CDR3 having the sequence SHY
and wherein the light chain variable region comprises
a CDR1 having the sequence RASQZ₁VVTZ₂VA, wherein Z₁ is N or G, and Z₂ is N or S, preferably wherein Z₁ is N and Z₂ is N or Z₁ is G and Z₂ is S;
a CDR2 having the sequence SASYLYS; and
a CDR3 having the sequence QQYZ₃SYPYT, wherein Z₃ is S or D.

2. The anti-fibronectin-EDA antibody or antigen binding fragment thereof according to claim 1, having a heavy chain variable region comprising the sequence of SEQ ID 1.

3. The anti-fibronectin EDA antibody or antigen binding fragment thereof according to claim 1, having a light chain variable region comprising the sequence of SEQ ID 2 or SEQ ID 3.

4. The antibody according to claim 1, comprising a constant region of a human antibody.

5. One or more nucleic acid molecules encoding an antibody or antigen binding fragment thereof according to claim 1.

6. A vector comprising the nucleic acid molecule of claim 5.

7. A cell comprising and/or producing an antibody or antigen binding fragment thereof according to claim 1.

8. A cell culture comprising a cell according to claim 7.

9. A method for producing an antibody or antigen binding fragment thereof according to claim 1, comprising culturing a cell culture according to claim 8 and harvesting said antibody or antigen binding fragment thereof from said culture.

10. A pharmaceutical composition comprising an antibody or antigen binding fragment thereof according to claim 1.

11-14. (canceled)

15. A method of treatment comprising administering to an individual in need thereof a therapeutically effective amount of the anti-fibronectin-EDA antibody or antigen binding fragment thereof according to claim 1.

16. The method of treatment according to claim 15, wherein said treatment is for the treatment, prevention, or prevention of the progression of fibrosis; for the treatment, prevention, or prevention of the progression of adverse cardiac remodeling, conditions resulting from or relating to myocardial infraction and/or pressure overload; or for improving angiogenesis.
